(19) United States
(12) Patent Application Publication
(10) Pub. No.: US 2014/0341863 A1
(43) Pub. Date: Nov. 20, 2014

(54) ADULT MESENCHYMAL STEM CELL (MSC) COMPOSITIONS AND METHODS FOR PREPARING THE SAME

(71) Applicant: NEOSTEM, INC., New York, NY (US)

(72) Inventors: Wayne Marasco, Wellesley, MA (US); Sean Hall, Bern (CH); Yajuan Jiang, Cambridge, MA (US)

(21) Appl. No.: 14/355,536

(22) PCT Filed: Oct. 31, 2012
(86) PCT No.: PCT/US12/62837

§ 371 Date: Apr. 30, 2014

Related U.S. Application Data
(60) Provisional application No. 61/554,290, filed on Nov. 1, 2011.

(51) Int. Cl. A61K 35/28; C12N 5/0775

(52) U.S. Cl. CPC A61K 35/28; C12N 5/0668
USPC 424/93.7; 435/325; 435/374

(57) ABSTRACT

The described invention provides a composition comprising mesenchymal progenitor cells (MPC) and processes for isolating or enriching the mesenchymal progenitor cells (MPCs) having a cell surface antigenic profile of CD34(−)/CD133(−)/CD45(−)/CD73(+)/CD90(+)/CD105(+)/CD44(−). The described invention also provides methods for differentiating the mesenchymal progenitor cells (MPCs) into various cell types.

### CROSS-REFERENCE TO RELATED APPLICATIONS

This application claims priority to U.S. provisional application No. 61/554,290, entitled, “Adult mesenchymal stem cell (MSC) compositions and methods for preparing the same,” filed Nov. 1, 2011, U.S. provisional Application No. 61/698,121, entitled, “Identification and isolation of small CD44 negative mesenchymal stem/progenitor cells from human bone marrow using elutriation and polychromatic flow cytometry,” filed Sep. 7, 2012; and International Patent Application No. PCT/US12/62837, entitled “Adult Mesenchymal Stem Cell (MSC) Compositions And Methods For Preparing The Same”, filed Oct. 31, 2012. Each of these applications is incorporated by reference herein in its entirety.

### FIELD OF INVENTION

The present invention relates to an adult progenitor/stem cell population having mesenchymal-like properties.

### BACKGROUND OF THE INVENTION

Mesenchymal stem/stromal cells (MSCs) are rare, non-hematopoietic adult stem cells originally found to reside in the stromal compartment of bone marrow (BM), the site of hematopoiesis. These fibroblast-like adult stem cells were first identified based on their ability to adhere to plastic surfaces. (Keating A., “Mesenchymal stromal cells,” Curr. Opin. Hematol., 13: 419-425 (2006); Bianco, P., Robey, P. G., Simmons, P. J., “Mesenchymal stem cells: revisiting history, concepts, and assays,” Cell Stem Cell. 2: 313-319 (2008)). These plastic adherent cells were further characterized by their: (1) ability to give rise to the colony forming unit-fibroblast (CFU-F); (2) ability to support hematopoiesis; and (3) osteogenic potential. (Friedenstein, A. J., Gorskaja, J. F., Kulagina, N. N., “Fibroblast precursors in normal and irradiated mouse hematopoietic organs,” Exp. Hematol., 4: 267-274 (1976); Friedenstein, A. J., Deriglasova, U. F., Kulagina, N. N. et al., “Precursors for fibroblasts in different populations of hematopoietic cells as detected by the in vitro colony assay method,” Exp. Hematol., 2: 83-92 (1974)). It is now known that MSCs exhibit spindle-shape morphology, display a clonogenic potential, are able to self-renew, and are characterized by their ability, under appropriate conditions, to differentiate into multiple cell types of mesenchymal lineages, such as adipocytes, osteoblasts, and chondrocytes in defined conditions in vitro.

Besides the BM, MSCs are also typically isolated from cartilage and muscle. (Pittenger, F. et al., “Multilineage potential of adult human mesenchymal stem cells,” Science, 284:143-147 (1999); Zuk, P, et al., “Multilineage cells from human adipose tissue—implications for cell-based therapies,” Tissue Eng., 7:211-228 (2001); Young H, et al., “Human reserve pluripotent mesenchymal stem cells are present in the connective tissues of skeletal muscle and dermis derived from fetal, adult, and geriatric donors,” Anat. Rec., 264:51-62 (2001)). MSC-like cells also have been identified from a number of postnatal organs, including dental pulp (Gronthos, S., Arthur, A., Bartold, P. M. et al., “A method to isolate and culture expand human dental pulp stem cells,” Methods Mol. Biol., 698: 107-121 (2011)), lung (Jarvinen, L., Badri, L., Wettlaufer, S. et al., “Lung resident mesenchymal stem cells isolated from human lung allografts inhibit T cell proliferation via a soluble mediator,” J. Immunol., 181: 4389-4396 (2008); Lama, V. N., Smith, L., Badri, L. et al., “Evidence for tissue-resident mesenchymal stem cells in human adult lung from studies of transplanted allografts,” J. Clin. Invest., 117: 989-996 (2007)), adipose tissue (Zannettino, A. C., Paton, S., Arthur, A. et al., “Multipotential human adipose-derived stromal stem cells exhibit a perivascular phenotype in vitro and in vivo,” J. Cell Physiol. 214: 413-421 (2008)); and peripheral blood. (Kassis, I., et al., “Isolation of mesenchymal stem cells from G-CSF-mobilized human peripheral blood using fibrin microbeads,” Bone Marrow Transplant, 37:967-976 (2006)). MSCs have also been isolated from neonatal tissue, such as Wharton's jelly (Wang, H., et al., “Mesenchymal stem cells in the Wharton's jelly of the human umbilical cord,” Stem Cells, 22:1330-1337 (2004)), placenta (Igura, K. et al., “Isolation and characterization of mesenchymal progenitor cells from chorionic villi of human placenta,” Cytotherapy, 6: 543-553 (2004); Fukuchi, Y, et al., “Human placenta-derived cells have mesenchymal stem/progenitor cell potential,” Stern Cells, 22:649-658 (2004)), umbilical cord (Erices, A, et al., “Mesenchymal progenitor cells in human umbilical cord blood,” Br. J. Haematol., 109: 235-242 (2000); Kim, H. S. et al., “Implication of NOD1 and NOD2 for the differentiation of multipotent mesenchymal stem cells derived from human umbilical cord blood,” PLoS One. 5: e15369 (2010)), and human fetal tissues (Campagnoli, C., et al., “Identification of mesenchymal stem/progenitor cells in human first-trimester fetal blood, liver, and bone marrow,” Blood, 98: 2396-2402 (2001)). While MSC-like cells are located throughout the body, the BM still represents the most extensively characterized source of MSC-like cells to date. (da Silva Meirelles, L., Caplan, A. I., Nardi, N. B., “In search of the in vivo identity of mesenchymal stem cells,” Stem Cells, 26:2287-2299 (2008)).

There is a need in the art for methods to isolate sufficient number of mesenchymal stem cells for differentiation and use. Isolation of MSCs sometimes depend on invasive procedures, such as bone marrow biopsy, with available donors. It is also difficult to maintain long term cultures free from bacterial or viral contamination.

Conventional Methods of Isolation

Diverse approaches to cell sorting have been described for isolation of specific types of cells that include positive or negative selection methods. For example, methods based on differences in specific immunological targets, such as cell surface markers (Smeland, E. B. et al., “Isolation and characterization of human hematopoietic progenitor cells: an effective method for positive selection of CD34+ cells,” Leukemia, 6(8):845-852 (1992)); methods based on receptor-ligand interactions (Chess, L. et al., “Inhibition of antibody-dependent cellular cytotoxicity and immunoglobulin synthesis by an antiserum prepared against a human B-cell Ia-like molecule,” J. Exp. Med., 144(1):113-122 (1976)); and methods based on differences in cell density (Boyum, A. “Separation of blood leucocytes, granulocytes and lymphocytes,” Tissue Antigens, 4(4):269-274 (1974)) have been used to isolate specific cells or subpopulations of cells.

In addition, electric field-induced rotation of cells using principles of AC electrokinetics has also been described for cell characterization, discrimination and sorting. (Arnold, W. M. et al., “Rotation of an isolated cell in a rotating electric field,” Naturwissenschaften, 69(6):297-298 (1982); Holzel, R. et al., “Dielectric properties of yeast cells as determined by electrorotation,” Biochim. Biophys. Acta, 1104(1):195-200 (1992); Wang, X. B. et al., “Changes in Friend murine erythroleukaemia cell membranes during induced differentiation determined by electrorotation,” Biochim. Biophys. Acta. 1193(2):330-344 (1994); Huang, H. et al., “Identification and characterization of hematopoietic stem cells from the yolk sac of the early mouse embryo,” Proc. Natl. Acad. Sci. USA., 90(21):10110-10114 (1993); Huang, Y. et al., “Differences in the AC electrodynamics of viable and non-viable yeast cells determined through combined dielectrophoresis and electrorotation studies,” Phys. Med. Biol., 37(7):1499-1517 (1992); Gascoyne, P. R. C. et al., “Dielectrophoretic separation of mammalian cells studied by computerized image analysis,” Meas. Sci. Technol., 3:439-445 (1992); Gascoyne, P. R. C. et al., “Manipulations of biological cells using travelling-wave dielectrophoresis,” Proc. IEEE 16th Annu Int. Conf. IEEE Engineering in Medicine and Biology Society; Baltimore, Md. pp. 772-773 (1994)). In electrorotation methods, cells become electrically polarized when subjected to an alternating current (AC) field, and an applied rotational field induces cell rotation. In non-homogenous fields, a rotating cell experiences a lateral dielectrophorectic (DEP) force, the frequency of which is a function of intrinsic electrical properties that in turn depend on cellular composition and organization. (Gascoyne, P. R. C. et al., “Dielectrophoretic separation of mammalian cells studied by computerized image analysis,” Meas. Sci. Technol., 3:439-445 (1992)). Cells differing in electrical polarizabilities can experience differential forces in non-homogenous electric fields. (Becker, F. F. et al., “Separation of human breast cancer cells from blood by differential dielectric affinity,” Proc. Natl. Acad. Sci. USA, 92: 860-865 (1995)). Dielectrophoretic force induced by electrorotation may result in separation of cells with differing properties. For example, differences in dielectric properties have been used to separate viable from non-viable yeast cells (Markx, G. H. et al., “Separation of viable and non-viable yeast using dielectrophoresis,” J. Biotechnol., 32(1): 29-37 (1994)); and to separate erythrocytes from bacterial suspensions (Wang, X.-B. et al., “Selective dielectophorectic confinement of bioparticles in potential energy wells,” J. Phys. D: Appl. Phys. 26: 1278-1285 (1993)). However, such methods are limited to separation of cell-types with significant differences in polarizabilities.

Field flow fractionation (FFF) methods have also been described for separation of matter using particle density, size, volume, diffusivity, and surface charge as parameters. (Reviewed in Giddings, J. C., “Field-flow fractionation: analysis of macromolecular, colloidal, and particulate materials,” Science, 260(5113): 1456-1465 (1993)). Such methods can be used to separate both biological and non-biological matter ranging in size from about 1 nm to more than about 100 μm. Separation by field flow fractionation results from differential retention in a stream of liquid flowing through a thin channel. FFF methods combine elements of chromatography, electrophoresis, and ultracentrifugation and utilize a flow velocity profile established in the thin channel when the fluid is caused to flow through the chamber. Such velocity profiles may be, for example, linear or parabolic. A field is then applied at right angles to the flow and serves to drive the matter into different displacements within the flow velocity profile. The matter being displaced at different positions within the velocity profile will be carried with the fluid flow through the chamber at differing velocities. Fields may be based on sedimentation, crossflow, temperature gradient, centrifugal forces, and the like. The technique suffers, however, from producing insufficiently pure cell populations, being too slow, or being too limited in the spectrum of target cells or other matter.

### Conventional Methods to Isolate MSCs

Mobilizing agents have been described in the isolation of MSCs from mobilized peripheral blood. (Kassis, I., et al., “Isolation of mesenchymal stem cells from G-CSF-mobilized human peripheral blood using fibrin microbeads,” Bone Marrow Transplant, 37:967-976 (2006)). WO 2011/069121 describes a method of administration of a mobilizing agent, such as granulocyte macrophage-colony stimulating factor (GM-CSF), granulocyte-colony stimulating factor (G-CSF), or AM3100 (plerixafor), combined with the optimal timing of collection and isolation of MSCs from mobilized peripheral blood following administration of the mobilizing agent, which is within 3 days or less to one day or less following administration.

Conventional strategies to isolate mesenchymal stem/progenitor cells (MSPCs) from mammalian tissues (in most cases unfractionated BM), which are used for both research and clinical purposes have relied solely on the capacity of these cells as contained within the stromal fraction to adhere to plastic, and thereafter the ability of these cells to expand in culture. Although such methods are relatively easier for isolation from human fluids during fetal development, isolation from adult body fluids is often complicated and not reproducible. Moreover, since the stromal fraction contains a plethora of non-specific cell types, a density gradient centrifugation step is utilized to enrich for a fraction of mononuclear cells, which are subsequently seeded onto regular plastic dishes. From this, a plating strategy is used whereby the non-adherent fraction is removed after 72 hours in culture and the remaining adherent cells give rise to a cell population that is given the appellation MSCs. However, this approach results in the selection of a heterogeneous population of starting cells. Furthermore, a plating strategy solely based on plastic adherence is limiting. Recently, other attempts have been made to isolate MSC-like cells using fluorescent activated cell sorting (FACS), and distinct markers found on the plasma membrane surface of MSCs were determined retrospectively post culture.

Counterflow centrifugal elutriation (CCE) represents an alternative approach for the separation and enrichment of cells based on their size and cell mass. (Banfalvi, G., “Cell cycle synchronization of animal cells and nuclei by centrifugal elutriation,” Nat. Protoc., 3: 663-673 (2008)). The concept of using elutriation for cell separation was first introduced by Lindahl. (Lindahl, P. E. “On counter streaming centrifugation in the separation of cells and cell fragments,” Biochim. Biophys. Acta., 21:411-415 (1956); Lindahl, P. E. “Principle of a counter-streaming centrifuge for the separation of particles of different sizes,” Nature, 161:648 (1948)). For clinical applications, CCE has been used in a variety of applications, such as: to enrich monocytes from a large volume of peripheral blood monocuclear cells (Wahl, L. M. et al., “Isolation of human mononuclear cell subsets by counterflow centrifugal elutriation (CCE). I. Characterization of B-lymphocyte-, T-lymphocyte-, and monocyte-enriched fractions by flow cytometric analysis,” Cell Immunol., 85:373-383 (1984)); to separate lymphocyte subpopulations from monocytes and granulocytes (Powell, D. J. Jr. et al., “Efficient clinical-scale enrichment of lymphocytes for use in adoptive immunotherapy using a modified counterflow centrifugal elutriation program,” Cytotherapy, 11:923-935 (2009)); to enrich tumor cells (Eifler, R. L. et al., “Enrichment of circulating tumor cells from a large blood volume using leukapheresis and elutriation: proof of concept,” Cytometry B Clin. Cytom., 80:100-111 (2011)); and to enrich progenitor cell populations from leukapheresis products (Dlubek, D. et al., “Enrichment of normal progenitors in counter-flow centrifugal elutriation (CCE) fractions of fresh chronic myeloid leukemia leukapheresis products,” Eur. J. Haematol., 68:281-288 (2002)).

Primitive progenitors within mouse BM have been isolated using CCE from the earliest elutriated fractions based on size and shown to contribute to lymphohematopoietic reconstitution (Jones, R. J. et al., “Characterization of mouse lymphohematopoietic stem cells lacking spleen colony-forming activity,” Blood, 88:487-491 (1996)), as well as multlineage engraftment to epithelial tissues in recipient mice (Krause, D. S. et al., Multi-organ, multi-lineage engraftment by a single bone marrow-derived stem cell,” Cell, 105:369-377 (2001)).

WO 2011/069117 describes a method for flow-rate separation of various stem cell populations from peripheral blood using elutriation (size-based separation) to negatively exclude cells based on size, and to separate peripheral blood into various fractions, each comprising a specific type of stem cell of interest. The method circumvents the need for using positive or negative selection methods, e.g. using cell-surface markers in fluorescence-activated cell sorting (FACS) or immunoselection methods to select or exclude a cell population. The method uses varying flow rates through elutriation devices, thereby separating the source peripheral blood sample into different fractions comprising, for example, cells smaller than VSELs, such as platelets collected at 35 ml/min or less; very small embryonic like stem cells (VSELs) collected at 50 ml/min to 70 ml/min; red blood cells with some hematopoeitic stem cells (HSCs) collected at 90 ml/min or less; pure HSCs are collected at 100 ml/min or less; and mesenchymal stem cells (MSCs) collected at 110 ml/min to 120 ml/min. Fractions obtained by elutriation can be further sorted based on positive and negative selection methods using known cell-surface markers. (WO 2011/069117), which is incorporated by reference herein.

Bone-Marrow Derived MSCs

BM MSCs are rare cells with an estimated frequency of one in every 10,000 to 100,000 nucleated BM cells. (Castro-Malaspina, H. et al., “Characterization of human bone marrow fibroblast colony-forming cells (CFU-F) and their progeny,” Blood, 56: 289-301 (1980)). Although MSCs have been suggested to occupy a perivascular niche in the BM (Bianco, P., “Minireview: The stem cell next door: skeletal and hematopoietic stem cell “niches” in bone,” Endocrinology. 2011; 152:2957-2962), there is a paucity of data pertaining to the physical characteristics and antigenic profile of the candidate progenitor population in vivo that gives rise to cultured MSCs. Moreover, cultured MSCs represent a heterogenous population of cells, since they are expanded from plastic-adherent cells obtained from unfractionated BM, which contains a number of other cell types that have the ability to adhere to a plastic surface, including endothelial cells, fibroblasts and monocytes. (Pevsner-Fischer, M. et al., “The origins of mesenchymal stromal cell heterogeneity,” Stem Cell Rev., 7: 560-568 (2011); Colter, D. C. et al., “Identification of a subpopulation of rapidly self-renewing and multipotential adult stem cells in colonies of human marrow stromal cells,” Proc. Natl. Acad. Sci. USA. 98: 7841-7845 (2001)).

Due to their relative ease of isolation from BM, expansion capability in vitro, and differentiation potential and immunomodulatory properties, BM-MSCs represent a promising cell-based therapy option for enhancing endogenous tissue repair and for suppressing autoimmunity. (Psaltis, P. J. et al., “Reparative effects of allogeneic mesenchymal precursor cells delivered transendocardially in experimental nonischemic cardiomyopathy,” JACC Cardiovasc Interv., 3: 974-983 (2010); Lee J. W. et al., “Potential application of mesenchymal stem cells in acute lung injury,” Expert Opin. Biol. Ther., 9: 1259-1270 (2009); Bartosh, T. J. et al., “Aggregation of human mesenchymal stromal cells (MSCs) into 3D spheroids enhances their antiinflammatory properties,” Proc. Natl. Acad. Sci. USA., 107: 13724-13729 (2010); Uccelli, A. et al., “Why should mesenchymal stem cells (MSCs) cure autoimmune diseases?” Curr. Opin. Immunol., 22: 768-774 (2010); Uccelli, A. et al. “Mesenchymal stem cells in health and disease,” Nat. Rev. Immunol., 8: 726-736 (2008); Le Blanc, K. et al., “Immunomodulation by mesenchymal stem cells and clinical experience,” J. Intern. Med., 262: 509-525 (2007)).

There is great interest in the in vivo identification of MSCs. Despite an emerging consensus regarding the topography of MSCs within the BM, there is a lack of agreement regarding their antigenic profile. (Bianco, P. “Minireview: The stem cell next door: skeletal and hematopoietic stem cell “niches” in bone,” Endocrinology, 152: 2957-2962 (2011); Keating, A., “Mesenchymal stromal cells: new directions,” Cell Stem Cell, 10: 709-716 (2012)). This is related to the fact that the antigenic profile of MSCs has been elucidated post-culture in an artificial environment. A unique signature of MSC markers is yet to be characterized. Morphological and functional criteria are used to identify MSCs. (Horwitz, E, et al., “Clarification of the nomenclature for MSC: the International Society for Cellular Therapy position statement,” Cytotherapy, 7:393 (2005); Dominici, M, et al., “Minimal criteria for defining multipotent mesenchymal stromal cells. The International Society for Cellular Therapy position statement,” Cytotherapy 8:315 (2006)).

Recently, using a limited set of markers, a number of studies have attempted to identify the immunophenotype of prospectively isolated cells that subsequently behave as MSCs in culture. (Keating, A., “Mesenchymal stromal cells: new directions,” Cell Stem Cell, 10: 709-716 (2012); Harichandan, A. et al., “Prospective isolation of human MSC,” Best Pract. Res. Clin. Haematol., 24: 25-36 (2011)). One of the markers, CD271, has received considerable attention, as it has been used to isolate cells from BM with MSC-like properties. (Tormin, A. et al., “CD146 expression on primary nonhematopoietic bone marrow stem cells is correlated with in situ localization,” Blood, 117: 5067-5077 (2011); Sacchetti, B. et al., “Self-renewing osteoprogenitors in bone marrow sinusoids can organize a hematopoietic microenvironment,” Cell, 131: 324-336 (2007); Churchman, S. M. et al., “Transcriptional profile of native CD271+ multipotential stromal cells: evidence for multiple fates, with prominent osteogenic and Wnt pathway signaling activity,” Arthritis Rheum., 64(8): 2632-2643 (2012)). However, there is a lack of accord on the antigenic profile even within this population. (Keating, A., “Mesenchymal stromal cells: new directions,” Cell Stem Cell, 10: 709-716 (2012)).

There is a growing need within the MSC field to develop expanded antibody panels (involving four or more surface markers) for prospective identification and purification of unique BM mesenchymal stem/progenitor cell (MSPC) populations, similar to the hematopoietic stem cell (HSC) field (Notta, F. et al., “Isolation of single human hematopoietic stem cells capable of long-term multilineage engraftment,” Science, 333: 218-221 (2011)) and endothelial progenitor cell (EPC) field (Mund, J. A. et al., “Flow cytometric identification and functional characterization of immature and mature circulating endothelial cells,” Arterioscler. Thromb. Vasc. Biol., 32: 1045-1053 (2012)). This approach would apply to therapeutic applications as well as for basic research, since the identity of the isolated MSPC population may be linked to its efficacy for specific clinical applications, such as regenerative or immunomodulatory therapies. (Keating, A., “Mesenchymal stromal cells: new directions,” Cell Stem Cell, 10: 709-716 (2012)).

However, most cell surface markers are non-homogenous and are found on other cell types, leading to isolation of a heterogeneous starting cell population. Regardless of the approach, the ensuing cell surface phenotype of an MSC is retrospectively determined post culture. Therefore, a distinct signature based on the cluster of differentiation (CD) molecules on the surface of the pure, initiating population of cells is not known. In addition, the size of the initiating cell population has not been determined due to the technical limitation of conventional isolation strategies.

Therefore, there is a need in the art for pure, initiating subpopulations of BM-MSCs with distinct antigenic signature profiles that can be used in cellular therapy and regenerative medicine. However, one of the main limiting factors in obtaining purified subpopulations of MSCs from the BM has been the conventional method of isolation of BM-MSCs.

The present invention describes an isolated morphologically and phenotypically distinct MSC subset population that lacks CD44 expression and exhibits a physical size that is more equivalent to HSCs than traditional MSCs, isolated using an elutriation process combined with magnetic cell depletion and polychromatic flow cytometry. The described process uses a 5-antibody marker panel including CD45, CD73, CD90, CD105 and CD44 that is commonly used for retrospective analysis of culture-expanded MSCs for prospective identification and isolation of BM MSPCs that display MSC activity in culture. MSC activity is found to reside in a population of small, CD45⁻CD73⁺CD90⁺CD105⁺ cells that lack expression of CD44s, the standard isoform of the CD44 cell adhesion molecule that is commonly used to identify MSCs. By CCE, these rare, small CD44⁻ cells co-fractionate with lymphocytes and are easily separated from plastic-adherent monocytes. Following expansion, the cells display phenotypic markers commonly associated with MSCs, including acquisition of CD44. These rare CD45⁻CD73⁺CD90⁺CD105⁺CD44⁻ MSPCs, which are between 5 and 12 microns in diameter, expand rapidly in culture and demonstrate tri-lineage mesenchymal differentiation potential into osteoblasts, chondrocytes and adipocytes in vitro. Thus, human BM contains a previously undescribed population of small MSPCs that lack expression of CD44. The described invention is directed to the isolation of rare CD45⁻CD73⁺CD90⁺CD105⁺CD44⁻ MSPCs to a high level of purity. The described MSPC subpopulation can be expanded ex vivo and can be used for clinical applications of tissue regeneration and immune modulation.

### SUMMARY OF THE INVENTION

The described invention provides compositions comprising mesenchymal progenitor cells (MPC) and processes for isolating or enriching the mesenchymal progenitor cells (MPCs) having a cell surface antigenic profile of CD34(−)/CD133(−)/CD45(−)/CD73(+)/CD90(+)/CD105(+)/CD44(−), methods for differentiating the mesenchymal progenitor cells (MPCs) into various cell types and their use in bone regeneration and spinal cord injury models.

According to one aspect, the described invention provides a composition comprising an isolated population of mesenchymal stem/progenitor cells (MSPCs), wherein the mesenchymal stem/progenitor cells (MSPCs) are of a cell surface antigenic profile CD34(−)/CD133(−)/CD45(−)/CD73(+)/CD90(+)/CD105(+)/CD44(−). According to one embodiment, at least 90% of the cells in the population are of the cell surface antigenic profile CD34(−)/CD133(−)/CD45(−)/CD73(+)/CD90(+)/CD105(+)/CD44(−). According to another embodiment, the composition further comprises a pharmaceutically acceptable carrier. According to another embodiment, size of the mesenchymal stem/progenitor cells (MSPCs) is from 5 μm to 10 μm. According to another embodiment, the mesenchymal stem/progenitor cells (MSPCs) are capable of being differentiated into ectodermal cells, mesodermal cells, or endodermal cells. According to another embodiment, the mesenchymal stem/progenitor cells (MSPCs) are capable of forming a three-dimensional spheroid. According to another embodiment, the ectodermal cells are capable of differentiation to neurons of a peripheral or central nervous system. According to another embodiment, the neurons express a neuronal marker β-Tubulin 3 (Tuj-1). According to another embodiment, the mesodermal cells are capable of differentiation to adipocytes, chondrocytes and osteoblasts. According to another embodiment, the mesodermal cells differentiated from the mesenchymal stem/progenitor cells (MSPCs) are capable of differentiation into ectodermal cells. According to another embodiment, the isolated mesenchymal stem/progenitor cells (MSPCs) can be expanded in a chemically defined medium. According to another embodiment, exposure of the mesenchymal stem/progenitor cells (MSPCs) to granulocyte-colony stimulating factor (G-CSF) results in appearance of a CD105(+)/CD44(+) cell population and a CD105(+)/CD44(−) cell population. According to another embodiment, the mesenchymal stem/progenitor cell population of a cell surface antigenic profile CD34(−)/CD133(−)/CD45(−)/CD73(+)/CD90(+)/CD105(+)/CD44(−) is purified from cellular components of a bone marrow aspirate acquired from a subject. According to another embodiment, the mesenchymal stem/progenitor cell population of a cell surface antigenic profile CD34(−)/CD133(−)/CD45(−)/CD73(+)/CD90(+)/CD105(+)/CD44(−) is purified from peripheral blood. According to another embodiment, the mesenchymal stem/progenitor cells are capable of being mobilized by a stem cell mobilizing agent from the bone marrow into peripheral blood. According to another embodiment, the stem cell mobilizing agent is at least one of G-CSF, GM-CSF, and plerixafor (AMD3100 According to another embodiment, the mesenchymal setm/progenitor cell population of a cell surface antigenic profile CD34(−)/CD133(−)/CD45(−)/CD73(+)/CD90(+)/CD105(+)/CD44(−) is purified from umbilical cord blood.

According to another aspect, the described invention provides a method for isolating and purifying a population of mesenchymal stem/progenitor cells (MSPCs) of a cell surface antigenic profile CD34(−)/CD133(−)/CD45(−)/CD73(+)/CD90(+)/CD105(+)/CD44(−), wherein the method is based on cluster of differentiation (CD) molecules on a surface of a pure initial population of cells, the method comprising: (a) acquiring a source of CD34(−)/CD133(−)/CD45(−)/CD73(+)/CD90(+)/CD105(+)/CD44(−) cells from a mammal; (b) depleting CD34-positive and CD133-positive cells from the cell source of (a) to obtain a first purified cell population of a cell surface antigenic profile CD34(−)/CD133(−); (c) fractionating the first purified cell population of (b) using antibodies against cell surface antigens CD45, CD73, and CD90 to obtain a second purified cell population of the cell surface antigenic profile CD34(−)/CD133(−)/CD45(−)/CD73(+)/CD90(+); and (d) further fractionating the second purified cell population of (c) using antibodies against cell surface antigens CD105 and CD44 to obtain a third purified cell population of the cell surface antigenic profile CD34(−)/CD133(−)/CD45(−)/CD73(+)/CD90(+)/CD105(+)/CD44(−); wherein the method does not employ adherent culture of an unfractionated mononuclear cell population. According to one embodiment of the method, depletion of the CD34-positive and the CD133-positive cells in (a) is carried out by using a magnetic bead selection system. According to another embodiment, steps (c) and (d) are performed with fluorescence-activated cell sorting (FACS). According to another embodiment, the method further comprises (e) cryopreserving the third purified cell population by admixing the third purified cell population with a cryoprotectant and storing the population at low temperature. According to another embodiment, the source of the CD34(−)/CD133(−)/CD45(−)/CD73(+)/CD90(+)/CD105(+)/CD44(−) cells is a bone marrow aspirate, a peripheral blood sample, or an umbilical cord. According to another embodiment, the source of the CD34(−)/CD133(−)/CD45(−)/CD73(+)/CD90(+)/CD105(+)/CD44(−) cells is a bone marrow aspirate. wherein the isolated and purified population of mesenchymal stem/progenitor cells (MSPCs) of a cell surface antigenic profile CD34(−)/CD133(−)/CD45(−)/CD73(+)/CD90(+)/CD105(+)/CD44(−) are purified from cellular components of a bone marrow aspirate acquired from a subject. According to another embodiment, the isolated population of mesenchymal stem/progenitor cells (MSPCs) of a cell surface antigenic profile CD34(−)/CD133(−)/CD45(−)/CD73(+)/CD90(+)/CD105(+)/CD44(−) is purified from peripheral blood. According to another embodiment, the population of mesenchymal stem/progenitor cells (MSPCs) of a cell surface antigenic profile CD34(−)/CD133(−)/CD45(−)/CD73(+)/CD90(+)/CD105(+)/CD44(−) is purified from umbilical cord blood.

According to another aspect, the described invention provides a method for obtaining an enriched population of mesenchymal stem/progenitor cells (MSPCs) using a size-based elutriation technique, wherein the size-based elutriation technique comprises flowing the sample obtained from a subject through a series of increasing flow rates in an elutriation device, and wherein each flow rate in the series of increasing flow rates collects a different population of cells in each flow rate fraction, the method comprising: (1) acquiring a sample comprising CD34(−)/CD133(−)/CD45(−)/CD73(+)/CD90(+)/CD105(+)/CD44(−) cells from a mammal; (2) flowing the sample at a first flow rate, wherein the first flow rate allows a first flow rate fraction comprising cells that are smaller than the mesenchymal stem/progenitor cells (MSPCs) in the sample to flow through the elutriation device and to be collected in a first cell collection bag of the elutriation device; (3) increasing the first flow rate to a second flow rate, wherein the second flow rate allows a second flow rate fraction comprising the mesenchymal stem/progenitor cells (MSPCs) in the sample to flow through the elutriation device and to be collected in a second cell collection bag of the elutriation device, and wherein the second flow rate fraction collected in the second cell collection bag comprises the enriched population of the mesenchymal stem/progenitor cells (MSPCs); (4) recirculating the sample comprising non-retained cells through the elutriation apparatus; and (5) optionally increasing the second flow rate to a third flow rate, wherein the third flow rate allows a third flow rate fraction comprising cells that are larger than the mesenchymal stem/progenitor cells (MSPCs) in the sample to flow through the elutriation device and to be collected in a third cell collection bag of the elutriation device. According to one embodiment of the method, the first flow rate ranges from about 20 ml/minute to about 40 ml/minute, and wherein the first flow rate fraction comprises a substantial number of platelets. According to another embodiment, the first flow rate is 20 ml/minute. According to another embodiment, the first flow rate is 30 ml/minute. According to another embodiment, the first flow rate is 40 ml/minute. According to another embodiment, the second flow rate to obtain the enriched population of the mesenchymal stem/progenitor cells (MSPCs) ranges from about 50 ml/minute to about 90 ml/minute. According to another embodiment, the second flow rate to obtain the enriched population of the mesenchymal stem/progenitor cells (MSPCs) is 50 ml/minute. According to another embodiment, the second flow rate to obtain the enriched population of the mesenchymal stem/progenitor cells (MSPCs) is 70 ml/minute. According to another embodiment, the second flow rate to obtain the enriched population of the mesenchymal stem/progenitor cells (MSPCs) is 90 ml/minute. According to another embodiment, the third flow rate to obtain the third flow rate fraction comprising cells that are larger than the mesenchymal stem/progenitor cells (MSPCs) is greater than 90 ml/minute. According to another embodiment, the third flow rate is greater than 90 ml/minute but less than 105 ml/minute. According to another embodiment, the third flow rate that is greater than 90 ml/minute but less than 105 ml/minute collects a population of cells comprising Hematopoietic Stem Cells (HSCs). According to another embodiment, wherein the method further comprises removing undesired cells or components. According to another embodiment, the method further comprises cryopreserving the enriched population of the mesenchymal stem/progenitor cells (MSPCs) by admixing the third purified cell population with a cryoprotectant and storing the cell population at a low temperature. According to another embodiment, the method further comprises: (6) depleting CD34-positive and CD133-positive cells from the collected mesenchymal stem/progenitor cells (MSPCs) of (5) to obtain a first purified cell population of a cell surface antigenic profile CD34(−)/CD133(−); (7) fractionating the first purified cell population of (6) using antibodies against cell surface antigens CD45, CD73, and CD90 to obtain a second purified cell population of the cell surface antigenic profile CD34(−)/CD133(−)/CD45(−)/CD73(+)/CD90(+); and (8) further fractionating the second purified cell population of (7) using antibodies against cell surface antigens CD105 and CD44 to obtain a third purified cell population of the cell surface antigenic profile CD34(−)/CD133(−)/CD45(−)/CD73(+)/CD90(+)/CD105(+)/CD44(−); wherein the method does not employ adherent culture of an unfractionated mononuclear cell population. According to another embodiment, the source of the CD34(−)/CD133(−)/CD45(−)/CD73(+)/CD90(+)/CD105(+)/CD44(−) cells is a bone marrow aspirate. wherein the isolated and purified population of mesenchymal stem/progenitor cells (MSPCs) of a cell surface antigenic profile CD34(−)/CD133(−)/CD45(−)/CD73(+)/CD90(+)/CD105(+)/CD44(−) are purified from cellular components of a bone marrow aspirate acquired from a subject. According to another embodiment, the isolated population of mesenchymal stem/progenitor cells (MSPCs) of a cell surface antigenic profile CD34(−)/CD133(−)/CD45(−)/CD73(+)/CD90(+)/CD105(+)/CD44(−) is purified from peripheral blood. According to another embodiment, the population of mesenchymal stem/progenitor cells (MSPCs) of a cell surface antigenic profile CD34(−)/CD133(−)/CD45(−)/CD73(+)/CD90(+)/CD105(+)/CD44(−) is purified from umbilical cord blood.

According to another aspect, the described invention provides a method for differentiating a mesenchymal stem/progenitor cells (MSPCs) into a neuron-like cell population, the method comprising: (a) culturing a population of mesenchymal stem/progenitor cells (MSPCs) of a cell surface antigenic profile CD34(−)/CD133(−)/CD45(−)/CD73(+)/CD90(+)/CD105(+)/CD44(−) in a chemically defined growth medium devoid of an animal serum; (b) generating a spheroid by plating the mesenchymal stem/progenitor cells (MPCs) on a low-attachment plate; (c) plating and culturing the mesenchymal stem/progenitor cells (MSPCs) from the spheroid in (b) in a defined medium for neuronal differentiation; and (d) obtaining the neuron-like cell population differentiated from the mesenchymal stem/progenitor cells (MSPCs). According to one embodiment, the neuron-like cell population expresses a neuronal marker β-Tubulin 3 (Tuj-1). According to another embodiment, culturing in (c) is continued at least for 21 days.

According to another aspect, the described invention provides aa method for treating a degenerative condition or a diseased tissue condition in a subject, the method comprising: (a) administering to the subject a therapeutically effective amount of the composition. According to one embodiment, the degenerative condition or diseased tissue condition is a neurodegenerative disease, a neurological injury, a musculoskeletal defect, or a combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows scatter plots of Fluorescence-Activated Cell Sorting (FACS) analyses for the prospective identification and isolation, using polychromtic flow cytometry/cell sorting, of small CD45⁻CD73⁺CD90⁺CD105⁺CD44⁻ human BM cells that are devoid of homing receptor CD44, and behave as MSCs in culture. Processed bone marrow was depleted of CD34 and CD133, and stained for the hematopoietic marker CD45 and mesenchymal markers CD73 and CD90. (A) Mononuclear cells (MNCs) were initially displayed on a Side Scatter (SSC) vs. Forward Scatter (FSC) color density plot of BM cells. (B) The initial SSC/FSC display in (A) was subgated onto an antigen plot identification of CD45⁻/7-AAD⁻ (live) cells (gate R3). (C) CD45⁻/7-AAD⁻ cells were further subgated to display of a cluster of CD73⁺CD90⁺ cells (gate R4) that are CD45-negative. (D) The CD73(+)/CD90(+)/CD45(−) cells were then displayed as a quadrant gate to identify CD105⁺CD44⁻ subset of cells. Sort window of CD105⁺CD44⁻ cells is indicated as R5. (E) Back-gating of the original CD45⁻CD73⁺CD90⁺CD105⁺CD44⁻ population revealed their location near the lymphocyte population within the SSC/FSC colour density plot (left panel) and standard sized flow cytometric beads confirmed their small size (about 5-10 μm) (right panel). (F) Representative image of typical CFU-F from CD45⁻CD73⁺CD90⁺CD105⁺CD44⁻ sorted cells. (G) higher power image of a single colony of the CD45⁻CD73⁺CD90⁺CD105⁺CD44⁻ sorted cells (4×). FACS sorted CD44⁻ cells were expanded in culture and demonstrate tri-lineage differentiation potential in vitro towards (H) adipocytes detected using Oil Red O stain for lipids, (I) osteoblasts detected using Alizarin Red S stain and (J) chondroblasts detected using safranin-O stain. Similar results were seen in 4 other BM samples from different donors.

FIG. 2 shows characteristics of elutriated fractions 70, 90, 110 and >110 from lysed BM. Distribution of: (A) viable nucleated cells; (B) percentage (%) of lymphocytes; (C) percentage (%) of monocytes; and (D) percentage (%) of granulocytes recovered from the various elutriated fractions. (E) Quantification of FACS sorted CD44⁻ cells from the 4 fractions. Peak recovery was found in fraction 90. (F) The percentage (%) of rare CD45⁻CD73⁺CD90⁺CD105⁺CD44⁻ cells from the various fractions when normalized to TNC demonstrates that fraction 90 contains the bulk of cells. (G) CD45⁻CD73⁺CD90⁺CD105⁺CD44⁻ cells from fraction 90 were sorted and the cumulative growth curve was calculated over 21 days after initial plating calculated as day 0. Data are presented as mean±SEM, n=5. *P<0.05 versus fraction 100-110 and fraction >110, using one-way analysis of variance followed by Newman-Keuls multiple comparison test.

FIG. 3 shows representative displays of SSC/FSC color density plots of the elutriated fractions 70 (A), 90 (B), 110 (C), and >110 (D). Representative images from one experiment. Similar results were seen in 4 other BM samples from different donors.

FIG. 4 shows representative displays of CD45⁻/7AAD density plot on the SSC/FSC color density plots used to identify CD45⁻/7-AAD⁻ (live) cells (gate R3) for the elutriated fractions 70 (A), 90 (B), 110 (C), and >110 (D). Representative images from one experiment. Similar results were seen in 4 other BM samples from different donors.

FIG. 5 shows representative displays of CD73/CD90 density plot on live CD45⁻ gated cells used to identify a cluster of live CD45⁻CD73⁺CD90⁺ cells (gate R4) for the elutriated fractions 70 (A), 90 (B), 110 (C), and >110 (D). Representative images from one experiment. Similar results were seen in 4 other BM samples from different donors.

FIG. 6 shows representative displays of the CD105⁺CD44⁻ plot on live CD45⁻CD73⁺CD90⁺ gated cells used to identify a cluster of CD105⁺CD44− cells (gate R5) for the elutriated fractions 70 (A), 90 (B), 110 (C), and >110 (D). Representative images from one experiment. Similar results were seen in 4 other BM samples from different donors.

FIG. 7 shows distribution of the CD45⁻CD73⁺CD90⁺ CD105⁺CD44⁻ population within the SSC/FSC colour density plot when back-gated onto a SSC-Height/FSC-Height dot plot for the elutriated fractions 70 (A), 90 (B), 110 (C), and >110 (D). Representative images from one experiment. Similar results were seen in 4 other BM samples from different donors.

FIG. 8 shows representative displays of SSC/FSC color density plots with flow cytometric beads of known size characterized based on side scatter (SSC) vs forward scatter (FSC) properties for the elutriated fractions 70 (A), 90 (B), and 110 (C).

FIG. 9 shows that CD34/CD133-depleted, FACS-sorted cells, which have a surface antigenic profile of CD34(−)/CD133(−)/CD45(−)/CD73(+)/CD90(+)/CD105(+)/CD44(−), form three-dimensional spheroids when plated on ultra-low attachment plates. (A) Culturing the CD34(−)/CD133(−)/CD45(−)/CD73(+)/CD90(+)/CD105(+)/CD44(−) cells in defined conditions for neuronal differentiation resulted in a change in phenotype and up-regulation of the neuronal marker Tuj-1 (beta III tubulin). (B) When spheroids were not cultured in neural differentiation media, no evidence of up-regulation in Tuj-1 expression was found in those cells.

FIG. 10 shows flow cytometric histograms of mesenchymal progenitor cells (MPCs) isolated by a conventional method using Ficoll/plastic or by using Fluorescence-Activated Cell Sorting (FACS) according to the described invention. The isolated cells were positive for CD105/CD44 and treatment of the cells with vehicle for 3 days did not alter the expression of CD105/CD44.

FIG. 11 shows single colour flow cytometric analysis of selected cell surface proteins on passage 3 culture expanded CD45⁻CD73⁺CD90⁺CD105⁺CD44⁻ cells (top panels in each row, FACS) compared to passage 3 donor matched MSCs isolated using conventional methods (bottom panels in each row, Ficoll). Representative images from one experiment. Similar results were seen in 4 other BM samples from different donors.

FIG. 12 shows that expanded CD44⁻ cells gain CD44 post culture, which is sensitive to recombinant human (rh) G-CSF treatment. (A) Representative flow cytometric density colour plots plots showing response of CD44⁻ FACS sorted cells (top panel) versus conventionally isolated MSCs (bottom panel) following treatment with 10 ng/mL of rhG-CSF over 3 days. While MSCs isolated via conventional methods using Ficoll/plastic adherence did not respond, the FACS-sorted cells were separated into two populations of cells based on CD44 expression. (B) FACS sorted cells show a loss of CD44 following treatment with rhG-CSF (CD44⁻ cells, black bar), whereas MSCs isolated using the conventional method of ficoll followed by plastic adherence did not (white bar). Bar graphs are depicted as mean±SEM from 3 independent experiments. *P<0.05 versus conventional MSCs using a unpaired t-test.

FIG. 13 shows the tri-lineage differentiation potential of small CD45⁻CD73⁺CD90⁺CD105⁺CD44⁻ cells obtained from Fraction 90 following expansion. (A) Growth of sorted CD44⁻ cells obtained from elutriation Fraction 90. Representative Giemsa-stained CFU-F colony at day 12 (left). Typical colony appearance by phase contrast (center). After 3 passages, elutriated/FACS sorted cells demonstrated a greater expansion capacity compared to conventional MSCs (right). (B-D) After passage 3, cells were placed in differentiation conditions in vitro to induce osteogenesis, chondrogeneis and adipogenesis. (B) Osteogensis was detected by staining cultures with 2% Alizarin Red S solution (pH=4.2). Representative image comparing osteogenesis cultures for CD44⁻ cells (left panel) to cultures of donor matched conventional MSCs (center panel). The monolayer of CD44− cells stained more densely with Alizain Red S. (C) To induce chondrogenesis, cell pellets were incubated with 10 ng/mL of recombinant human TGF-β3. Chondrogenesis was detected following staining with safranin-O. Representative image of CD44⁻ pellet (left panel) demonstrates more intense staining compared to donor matched conventional MSCs (right panel). (D) Adipogenesis was detected following staining of cultures with Oil Red O, which detects lipids. Representative image of CD44⁻ culture (left panel) compared to compared to donor matched conventional MSCs isolated (center panel) is shown. Spectrophotometric quantification of adipogenesis following isopropanol extraction of Oil Red O (far right). Data are presented as mean±SEM, n=4-5. *P<0.05 versus conventional isolated MSCs, using an unpaired t-test.

FIG. 14 shows the identification and isolation of CD44⁺ cells in fractionated BM using CCE followed by FACS. (A) CD73/CD90 pseudocolor plot from the live/CD45⁻ cell gate (not shown) shows a cluster of CD73⁺CD90⁺ cells (gate R4), and an additional gate, R1 added to display CD73⁺CD90⁻ cells. (B) The R1 sort window was further subgated onto a CD105/CD44 antigen plot, showing CD105⁺CD44⁻ (gate R29) and CD105⁻CD44⁺ (gate R32) subpopulations. (C) Representative phase contrast image show that CD45⁻CD73⁺CD90⁻CD105⁻CD44⁺ cells (gate R32) sorted from Fraction 90 were expandable but grew as a spheroid (top panel, day 24) compared to sorted CD45⁻CD73⁺CD90⁺CD105⁺CD44⁻ cells (bottom panel, day 9), which grow as a monolayer. Following EDTA treatment and mechanical disruption of CD45⁻CD73⁺CD90⁻CD105⁻CD44⁻ spheroids, the cells were able to be expanded under defined conditions in vitro, and differentiated to (D) adipocytes detected using Oil Red O stain and (E) chondroblasts using safranin-O stain. Data representative of one experiment.

### DETAILED DESCRIPTION OF THE INVENTION

### Glossary

The term “active” as used herein refers o the ingredient, component or constituent of the composition of the described invention responsible for the intended therapeutic effect.

The term “administer” as used herein means to give or to apply. The term “administering” as used herein includes in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, topical application, or parenterally.

The term “adipocyte” as used herein refers to a cell, which is located between tissues or forms fat tissue as areolar tissue or a group along capillary blood vessels, and which contains a large amount of lipid. The term “adipocyte” as used herein include both yellow adipocyte and a brown adipocyte.

As used herein, the term “antibody” includes, by way of example, both naturally occurring and non-naturally occurring antibodies. Specifically, the term “antibody” includes polyclonal antibodies and monoclonal antibodies, and fragments thereof. Furthermore, the term “antibody” includes chimeric antibodies and wholly synthetic antibodies, and fragments thereof

Antibodies are serum proteins the molecules of which possess small areas of their surface that are complementary to small chemical groupings on their targets. These complementary regions (referred to as the antibody combining sites or antigen binding sites) of which there are at least two per antibody molecule, and in some types of antibody molecules ten, eight, or in some species as many as 12, may react with their corresponding complementary region on the antigen (the antigenic determinant or epitope) to link several molecules of multivalent antigen together to form a lattice.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

Human antibodies show two kinds of light chains, κ and λ; individual molecules of immunoglobulin generally are only one or the other. In normal serum, 60% of the molecules have been found to have κ determinants and 30 percent λ. Many other species have been found to show two kinds of light chains, but their proportions vary. For example, in the mouse and rat, λ chains comprise but a few percent of the total; in the dog and cat, κ chains are very low; the horse does not appear to have any κ chain; rabbits may have 5 to 40% λ, depending on strain and b-locus allotype; and chicken light chains are more homologous to λ than κ.

In mammals, there are five classes of antibodies, IgA, IgD, IgE, IgG, and IgM, each with its own class of heavy chain—α (for IgA), δ (for IgD), ∈ (for IgE), γ (for IgG) and μ (for IgM). In addition, there are four subclasses of IgG immunoglobulins (IgG1, IgG2, IgG3, IgG4) having γ1, γ2, γ3, and γ4 heavy chains respectively. In its secreted form, IgM is a pentamer composed of five four-chain units, giving it a total of 10 antigen binding sites. Each pentamer contains one copy of a J chain, which is covalently inserted between two adjacent tail regions.

All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur, and that this phenomenon gives rise to cross-reactions. Cross reactions are of major importance in understanding the complementarity or specificity of antigen-antibody reactions. Immunological specificity or complementarity makes possible the detection of small amounts of impurities/contaminations among antigens.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (VH) and light (Vκ and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human λ light chains with the heavy chain variable region (VH) domain of a mouse monoclonal antibody reactive with an antigen of interest; (ii) selecting half-human Fabs on that antigen (iii) using the selected λ light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ antibody molecule in the mouse myeloma.

The term “antigen” and its various grammatical forms refers to any substance that can stimulate the production of antibodies and can combine specifically with them. The term “antigenic determinant” or “epitope” as used herein refers to an antigenic site on a molecule.

The terms “apheresis”, “hemapheresis”, and “pheresis” refer to the process or procedure in which blood is drawn from a donor subject and separated into its components, some of which are retained, such as plasma, platelets and/or stem cell populations, and the remainder returned by transfusion to the donor subject. The forms of apheresis include: Plasmapheresis—to harvest plasma (the liquid part of the blood); Leukapheresis—to harvest leukocytes (white blood cells); Granulocytapheresis—to harvest granulocytes (neutrophils, eosinophils, and basophils); Lymphocytapheresis—to harvest lymphocytes; Lymphoplasmapheresis—to harvest lymphocytes and plasma; Plateletpheresis (thrombocytapheresis)—to harvest platelets (thrombocytes). Apheresis takes longer than a whole blood donation. A whole blood donation takes about 10-20 minutes to collect the blood, while an apheresis donation may take about 1-2 hours.

The term “apheresis product” as used herein refers to the heterogeneous population of cells collected from the process of apheresis. According to one embodiment, the cells present in an apheresis product may be separated using elutriation.

The term “bone marrow-derived” used in connection with a stem cell refers to a stem cell which is mobilized from the bone marrow to the peripheral blood, and can include stem cells which have migrated from the BM. In some embodiments, BM-derived stem cells in the peripheral blood include stem cells, which have proliferated in the bone marrow prior to migration to the peripheral blood, or alternatively stem cells which have proliferated in the peripheral blood after migration from the bone marrow. In some embodiments, the number of circulating BM-stem cells can be increased in the peripheral blood by contacting the peripheral blood with a mobilizing agent in vivo according to the methods as disclosed herein.

The term “cardiovascular disorder” as used herein refers to a disorder or condition that affects the heart and blood vessels. Examples of cardiovascular disorders susceptible to treatment with the composition of the described invention include, without limitation, arterial enlargements, arterial narrowing, peripheral artery disease, atherosclerosis, hypertension, angina, irregular heart rates (arrhythmia), inappropriate rapid heart rate (tachycardia), inappropriate slow heart rate (bradycardia), angina pectoris, heart attack, myocardial infarction, transient ischemic attacks, heart enlargement, heart failure congested heart failure, heart muscle weakness, heart valve leaks, heart valve stenosis (failure to open), strokes, chronic renal insufficiency, and diabetic or hypertensive nephropathy.

The term “carrier” as used herein describes a material that does not cause significant irritation to an organism and does not abrogate the biological activity and properties of the cell of the composition of the described invention. Carriers must be of sufficiently high purity and of sufficiently low toxicity to render them suitable for administration to the mammal being treated. The carrier can be inert, or it can possess pharmaceutical benefits. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components.

The term “cell fractionation” as used herein refers to a broad set of techniques that either separate or isolate cells from a heterogeneous population of cells, including, but not limited to, differential centrifugation, flow cytometry, and fluorescence-activated cell sorting (FACS).

The term “cell surface antigen” as used herein refers to a cell-associated component on the outside surface of the cell that can behave as an antigen without disrupting the integrity of the membrane of the cell expressing the antigen.

The term “chemically defined medium” as used herein refers to a nutritive medium used for cell culture substantially free of animal serum substances, and where all components and their concentration are known and described.

The term “CD” or “cluster of differentiation” as used herein refers to a defined subset of cellular surface receptors (epitopes) that identify a cell type and a stage of differentiation, and which are recognized by antibodies.

The term “colony stimulating factor” refers to a cytokine responsible for controlling the production of white blood cells. Types of colony stimulating factors include granulocyte colony stimulating factor (G-CSF), macrophage colony stimulating factor (M-CSF), and granulocyte macrophage colony stimulating factor (GM-CSF).

The term “compatible” as used herein means that the active ingredients of such a composition are capable of being combined with each other in such a manner so that there is no interaction that would substantially reduce the efficacy of each active ingredient or the composition under ordinary use conditions.

The term “component” as used herein refers to a constituent part, element or ingredient.

The terms “composition” and “formulation” are used interchangeably herein to refer to a product of the described invention that comprises all active and inert ingredients. The terms “pharmaceutical formulation” or “pharmaceutical composition” as used herein refer to a formulation or composition that is employed to prevent, reduce in intensity, cure or otherwise treat a target condition or disease.

The term “condition”, as used herein, refers to a variety of health states and is meant to include disorders or diseases caused by any underlying mechanism or disorder, injury, and the promotion of healthy tissues and organs.

The term “contact” and all its grammatical forms as used herein refers to a state or condition of touching or of immediate or local proximity.

The term “cryopreserve” or its various grammatical forms as used herein refers to preserving cells for long term storage in a cryoprotectant at a low temperature. The term “cryoprotectant” as used herein refers to an agent that minimizes ice crystal formation in a cell or tissue, when the cell or tissue is cooled to subzero temperatures and results in no substantially damage to the cell or tissue after warming, in comparison to the effect of cooling without cryoprotectant.

The term “degenerative condition” as used herein refers to a disorder or condition characterized by a deterioration in function resulting from a retrogressive pathologic change in cells or tissues, in consequence of which the function(s) of the cells or tissues is (are) impaired or destroyed.

The term “differentiation” as used herein refers to the cellular development of a cell from a primitive stage to a mature formation that is associated with the expression of characteristic set of cell surface antigenic markers. Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway (“terminally differentiated cell”).

The term “diseased tissue condition” as used herein refers to a disorder or condition characterized by an interruption, cessation, or disorder of body function, system or organ characterized by at least two of the following criteria: recognized etiologic agent(s), identifiable group of signs and symptoms, or consistent anatomic alterations. Examples of diseased tissue conditions susceptible to treatment with the composition of the described invention, include, without limitation, a neurodegenerative disease, a neurological injury, a cardiovascular disorder, and a musculoskeletal defect.

The term “drug” as used herein refers to a therapeutic agent or any substance, other than food, used in the prevention, diagnosis, alleviation, treatment, or cure of disease.

The term “ectoderm” refers to the outermost of the three primitive germ layers of the embryo. The other two layers are the “mesoderm” (middle layer) and “endoderm” (inside layer). Following gastrulation, various cell lineages are derived from these three primary cell types.

The term “ectodermal cells” as used herein refers to cells possessing the characteristics of the embryonic ectoderm or cells derived from the embryonic ectoderm. Examples of ectodermal cells include, but are not limited to, nail cells, hair cells, tooth cells, and the cells of a central and peripheral nervous system.

The term “effective amount” refers to the amount necessary or sufficient to realize a desired biologic effect.

The term “efficacy” as used herein refers to the property of the compositions of the present invention to achieve the desired response, and “maximum efficacy” refers to the maximum achievable effect.

The term “elutriation” as used herein refers to a process for separating lighter particles from heavier ones using a vertically-directed stream of a gas or liquid (usually upwards). It is a noninvasive method for separating large numbers of cells on the basis of their size and mass that allows for separating mixed populations of cells at different stages of the cell division cycle without perturbing cell metabolism or using synchronizing agents.

The term “endodermal cells” as used herein refers to cells possessing the characteristics of the embryonic endoderm or cells derived from the embryonic endoderm. Examples of endodermal cells include, but are not limited to, the cells of thyroid gland, liver, intestine, pancreas, spleen and lung.

The terms “enriching” or “enriched” or “enrich” are used interchangeably herein and mean that the yield (i.e., fraction) of cells of one type is increased by at least 10% over the fraction of cells of that type in the starting culture or preparation.

The term “fractionation” as used herein refers to the act or process of separating a population of heterogeneous stem cells into its component relatively homogeneous stem cell populations.

The term “immunoselection” as used herein refers to a process where cells, e.g., stem cells, are labeled using monoclonal antibodies, which bind to cell markers, and whereby the cells are separated by selecting for the bound antibody, which is tagged with fluorescence, or magnetic particles.

The term “isolated” is used herein to refer to material, such as, but not limited to, a nucleic acid, peptide, polypeptide, protein, or cell, which is: (1) substantially or essentially free from components that normally accompany or interact with it as found in its naturally occurring environment. The term “isolated population,” with respect to an isolated population of cells as used herein, refers to a population of cells that has been removed and separated from a mixed or heterogeneous population of cells. According to some embodiments, an isolated population is a substantially pure population of cells as compared to the heterogeneous population from which the cells were isolated or enriched from. In some embodiments, the isolated population is an isolated population of reprogrammed cells, which is a substantially pure population of reprogrammed cells, as compared to a heterogeneous population of cells comprising reprogrammed cells and cells from which the reprogrammed cells were derived.

The term “hematopoietic stem cells,” also referred to as “HSCs,” refers to all stem cells or progenitor cells found inter alia in bone marrow and peripheral blood that are capable of differentiating into any of the specific types of hematopoietic or blood cells, such as erythrocytes, lymphocytes, macrophages and megakaryocytes. HSCs are reactive with certain monoclonal antibodies which are specific for hematopoietic cells, for example, monoclonal antibodies that recognize CD34.

The term “mammal” as used herein refers to an animal species of mammalian origin, including but not limited to, a mouse, a rat, a cat, a goat, sheep, horse, hamster, ferret, platypus, pig, a dog, a guinea pig, a rabbit and a primate, such as, for example, a monkey, ape, or human.

The term “marker” as used herein refers to a characteristic and/or phenotype of a cell, whether morphological, functional or biochemical (enzymatic) characteristics particular to a cell type, or molecules expressed by the cell type. Exemplary markers include, but are not limited to proteins, e.g. possessing an epitope for antibodies or other binding molecules available in the art; any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers, which vary depending on the cell type, can be used for selection of cells comprising characteristics of interest, and may be detected by any method available to one of skill in the art.

The term “mesenchymal stem cells (MSCs)” as used herein refers to pluripotent stem cells capable of differentiating into more than one specific type of mesenchymal or connective tissue (i.e., tissues of the body which support specialized elements; e.g., adipose, osseous, stroma, cartilaginous, elastic and fibrous connective tissues). Human mesenchymal stem cells (hMSCs) are reactive with certain monoclonal antibodies, known as SH2, SH3 and SH4. (See U.S. Pat. No. 5,486,359, which is incorporated by reference herein in its entirety). MSCs can be differentiated from HSCs based on their immunospecific profiles, with MSCs being SH2+/CD14− and human HSCs SH2−/CD14+. For purposes of identification, human MSCs can be identified based on (i) phenotypic marker expression of CD34−, CD45−, CD90+, CD105+ and CD44+, (ii) functional phenotype, including the ability to form colony forming units in a CFA assay as disclosed in the Examples herein, and the ability to differentiate into tissues which support specialized elements, including but not limited to, chondrocytes, cartilage and adipocytes. Other markers expressed by MSCs are known in the art and include without limitation CD71, CD73, Stro-1, and CD166, and CD271.

The term “mesenchymal stem/progenitor cells (MSPCs)” as used herein refers to non-blood adult stem cells that are smaller than MSCs and comprise a distinct set of cell surface markers. MSPCs are also capable of differentiating along a minimum of three lineages (osteogenic, chondrogenic and adipogenic).

The term “mesodermal cells” as used herein refers to cells possessing the characteristics of the embryonic mesoderm or cells derived from the embryonic mesoderm. Examples of mesodermal cells include, but are not limited to, bone cells, muscle cells, connective tissue cells, and the cells of the middle layer of the skin.

The term “mobilization” as used herein refers to the process whereby the cells leave the bone marrow and enter the blood. Mobilization may be effectuated by a combination of chemoattractants (e.g., cytokines) and loss of adhesiveness of pools or populations of stem cells residing in stem cell niches in peripheral tissues and the bone marrow.

The terms “mobilized source sample” as used herein refers to a source sample comprising target stem cells obtained from a subject where the subject has been administered a mobilizing agent to enhance the number of stem cells in the source sample by increasing the migration of stem cells from the bone marrow (e.g., increasing BM-derived stem cells) into the peripheral blood, or increasing the proliferation of stem cells present in the peripheral blood (e.g. increasing number of PB-derived stem cells). Mobilization may be effectuated in a subject by administering an effective amount of a mobilizing agent, e.g., by a combination of chemoattractants (e.g., cytokines) and loss of adhesiveness of pools or populations of stem cells residing in stem cell niches in peripheral tissues and the bone marrow. An “effective amount” is an amount of a mobilizing agent sufficient to effect a significant increase in the number and/or frequency of stem cells in the peripheral blood. An effective amount can be administered in one or more administrations, applications or dosages. A therapeutically effective amount of a mobilizing agent depends on the mobilizing agent selected. The mobilizing agent, such as G-CSF or GM-CSF, can be administered from one or more times per day to one or more times per week; including once every other day. The skilled artisan will appreciate that certain factors may influence the dosage and timing required to effectively treat a subject, including but not limited to, previous treatments, the general health and/or age of the subject, and whether other diseases are present. Moreover, treatment of a subject with a therapeutically effective amount of the G-CSF or GM-CSF, as commonly known by persons of ordinary skill in the art, can include a single treatment or a series of treatments.

The term “musculoskeletal defects” or “musculoskeletal disorder” as used herein refers to a disease or condition, which affects the body's musculoskeletal system, such as muscles, joints, tendons, ligaments, and bursa (small fluid-filled sac made of white fibrous tissue and lined with synovial membrane). Examples of musculoskeletal defects susceptible to treatment with the composition of the described invention include, without limitation, osteoporosis, rheumatoid arthritis; degenerative arthritis, degenerative spine disease, degenerative disc disease, muscular dystrophy, fibromyalgia, dermatomyositis, and polymyositis.

The term “negative selection” as used herein refers to targeting unwanted or non-target stein cells for depletion, e.g., using monoclonal antibodies to specific cell surface antigens. In negative selection, desired cells or target cells are not labeled with antibody.

The term “neurodegenerative disease” as used herein refers to a condition or disorder characterized by loss or degeneration of neurons. Examples of neurodegenerative diseases, susceptible to treatment with the composition of the described invention, include, without limitation, multiple sclerosis, Parkinson's disease, Huntington's disease, Alzheimer's disease, amyotrophic lateral sclerosis, and spinal muscular atrophy.

The term “neurological injury” as used herein refers to an insult to an element of the central or peripheral nervous system. Neurological injuries can be derived from a physical (including mechanical, electrical, or thermal), ischemic, hemorrhagic, chemical, biological or biochemical insult. Examples of neurological injuries, susceptible to treatment with the composition of the described invention, include, without limitation, cerebrovascular insufficiency, focal or diffuse brain trauma, diffuse brain damage, traumatic neuropathies (for example, compression, crush, laceration and segmentation neuropathies), cerebral ischemia or infarction (for example, embolic occlusion, thrombotic occlusion), reperfusion following acute ischemia, perinatal hypoxic-ischemic injury, intracranial hemorrhage of any type (for example, epidural, subdural, subarachnoid, and intracerebral), and tumors and other neoplastic lesions affecting the central nervous system and peripheral nervous system.

The term “osteoblast” or “osteoblast cell” as used herein refers to a bone progenitor cell, which has the capacity to form, or to contribute to the formation of new bone tissue. Osteoblasts include osteocytes and more immature osteoblast lineage cells.

The phrases “parenteral administration” and “administered parenterally” as used herein mean modes of administration other than enteral and topical administration, usually by injection, and include, without limitation, intravenous, intramuscular, intraarterial, intrathecal, intraventricular, intracapsular, intraorbital, intracardiac, intradermal, intraperitoneal, ranstracheal, subcutaneous, subcuticular, intraarticular, sub capsular, subarachnoid, intraspinal, intracerebro spinal, and intrasternal injection and infusion. The phrases “systemic administration,” “administered systemically,” “peripheral administration” and “administered peripherally” as used herein mean the administration of a target stem cell population or differentiated progeny thereof and/or their progeny and/or compound and/or other material other than directly into the subject, such that it enters the animal's system and, thus, is subject to metabolism and other like processes, for example, subcutaneous or intravenous administration.

The term “peripheral blood” as used herein refers to whole blood obtained from a subject. The term “peripheral blood-derived” used in connection with a stem cell refers to a stem cell which is mobilized from the peripheral blood only, and can include expansion or proliferation of the stem cell in the peripheral blood. In some embodiments, the number of circulating stem cells can be increased in the peripheral blood by contacting the peripheral blood with a mobilizing agent, either in vivo or ex vivo, according to the methods as disclosed herein.

The term “positive selection” as used herein refers to a method where the desired stem cells are targeted for selection, e.g., using a monoclonal antibody to a specific cell surface antigen on the desired or target stem cell.

The term “prevent” as used herein refers to the keeping, hindering or averting of an event, act or action from happening, occurring, or arising.

The term “progenitor cell” as used herein refers to a stem cell that has a cellular phenotype that is more primitive (e.g., is at an earlier step along a developmental pathway or progression than is a fully differentiated cell) relative to a cell to which it can give rise by differentiation. Often, progenitor cells have significant or very high proliferative potential. Progenitor cells can give rise to multiple distinct differentiated cell types or to a single differentiated cell type, depending on the developmental pathway and on the environment in which the cells develop and differentiate.

The term “purify” as used herein means to separate from unwanted components.

The term “repair” as used herein as a noun refers to any correction, reinforcement, reconditioning, remedy, making up for, making sound, renewal, mending, patching, or the like that restores function. When used as a verb, it means to correct, to reinforce, to recondition, to remedy, to make up for, to make sound, to renew, to mend, to patch or to otherwise restore function. According to some embodiments “repair” includes full repair and partial repair.

The term “recombinant” as used herein refers to a substance produced by genetic engineering.

The term “reduced” or “to reduce” as used herein refer to a diminution, a decrease, an attenuation or abatement of the degree, intensity, extent, size, amount, density or number.

The term “regeneration” means regrowth of a cell population, organ or tissue after disease or trauma.

The terms “renewal” or “self-renewal” or “proliferation” are used interchangeably herein to refer to a process of a cell making more copies of itself (e.g., duplication of the cell). According to some embodiments, reprogrammed cells are capable of renewing themselves by dividing into the same undifferentiated cells (e.g., pluripotent or non-specialized cell type) over long periods, and/or many months to years. In some instances, proliferation refers to the expansion of reprogrammed cells by the repeated division of single cells into two identical daughter cells.

The terms “separation” or “selection” as used herein refer to the act of isolating different cell types into one or more populations and collecting the isolated population as a target cell population enriched in a specific target stem cell population. Selection can occur using positive selection for the enriched cell population or negative selection to discard non-target cell populations.

The term “similar” is used interchangeably with the terms analogous, comparable, or resembling, meaning having traits or characteristics in common.

The term “spheroid” as used herein refers to spherical, heterogeneous aggregates of proliferating, quiescent, and necrotic cells in culture that retain three-dimensional architecture and tissue-specific functions.

The term “stem cells” as used herein refers to undifferentiated cells having high proliferative potential with the ability to self-renew that can generate daughter cells that can undergo terminal differentiation into more than one distinct cell phenotype, and includes traditional stem cells, progenitor cells, preprogenitor cells, reserve cells, and the like. The terms “stem cell” and “progenitor” are used interchangeably herein to refer to an undifferentiated cell, which is capable of proliferation and of giving rise to more progenitor cells having the ability to generate a large number of mother cells that can in turn give rise to differentiated, or differentiable, daughter cells. The daughter cells themselves can be induced to proliferate and produce progeny that subsequently differentiate into one or more mature cell types, while also retaining one or more cells with parental developmental potential. Thus, the term “stem cell” refers to a cell with the capacity or potential, under particular circumstances, to differentiate to a more specialized or differentiated phenotype, and which retains the capacity, under certain circumstances, to proliferate without substantially differentiating. In one embodiment, the term “stem/progenitor cell” refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell, which itself is derived from a multipotent cell, and so on. While each of these multipotent cells may be considered stem cells, the range of cell types to which each can give rise may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “sternness.” Self-renewal is the other classical part of the stem cell definition. In theory, self-renewal can occur by either of two major mechanisms. Stern cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, which is referred to as “dedifferentiation.”

Exemplary stem cells include embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including method for isolating and culturing them, may be found in, among other places, e.g., Embryonic Stem Cells, Methods and Protocols, Turksen, ed., Humana Press, 2002; Weisman et al., Annu Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); Atala et al., particularly Chapters 33 41; and U.S. Pat. Nos. 5,559,022, 5,672,346 and 5,827,735. Descriptions of stromal cells, including methods for isolating them, may be found in, among other places, Prockop, Science, 276:71 74, 1997; Theise et al., Hepatology, 31:235 40, 2000; Current Protocols in Cell Biology, Bonifacino et al., eds., John Wiley & Sons, 2000 (including updates through March, 2002); and U.S. Pat. No. 4,963,489, each of which is incorporated herein by reference.

Stem cells can be “totipotent,” “pluripotent” and “multipotent”. The term “totipotent” refers to a stem cell that can give rise to any tissue or cell type in the body. “Pluripotent” stem cells can give rise to any type of cell in the body except germ line cells. Stem cells that can give rise to a smaller or limited number of different cell types are generally termed “multipotent.” Thus, totipotent cells differentiate into pluripotent cells that can give rise to most, but not all, of the tissues necessary for fetal development. Pluripotent cells undergo further differentiation into multipotent cells that are committed to give rise to cells that have a particular function. For example, multipotent hematopoietic stem cells give rise to the red blood cells, white blood cells and platelets in the blood. The terms “pluripotency” or a “pluripotent state” as used herein refer to a cell with the ability to differentiate into all three embryonic germ layers: endoderm (gut tissue), mesoderm (including blood, muscle, and vessels), and ectoderm (such as skin and nerve), and that typically has the potential to divide in vitro for a long period of time, e.g., greater than one year or more than 30 passages. Pluripotent cells are characterized primarily by their ability to differentiate to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. According to some embodiments, a pluripotent cell is an undifferentiated cell. The term “multipotent” when used in reference to a “multipotent cell” refers to a cell that is able to differentiate into some, but not, all of the cells derived from all three germ layers. For example, a multipotent blood stem cell can form the many different types of blood cells (red, white, platelets, etc.), but it cannot form neurons. A multipotent cell is a partially differentiated cell. Multipotent cells are well known in the art, and examples of multipotent cells include adult stem cells, such as for example, hematopoietic stem cells and neural stem cells.

The term “stem cell mobilizing agent” refers to an agent used to increase stem cell yield by mobilizing stem cells from the bone marrow into peripheral blood for collection. Examples include, without limitation, Granulocyte colony-stimulating factor (G-CSF), Pegylated G-CSF (pegfilgrastim, Neulasta, Amgen, Inc.), a longer-lasting variant of G-CSF, GM-CSF (sargramostim, Leukine, Bayer Healthcare Pharmaceuticals); Plerixafor (AMD3100, Genzyme Genzyme Corporation, Cambridge, Mass.), a specific inhibitor of CXCR4, SB-251353, an analog of GRO-β, a human CXC chemokine involved in directing the movement of stem cells and leukocytes; recombinant thyroid peroxidase (rhTPO), recombinant parathyroid hormone (rPTH), recombinant methionyl human SCF (ancestim, Stemgen, Amgen, Inc.), and erythropoietin. See, e.g., Bensinger, W. et al., Bone Marrow Transplantation 43: 181-195 (2009)).

The terms “subject” and “individual” are used interchangeably herein, and refer to an animal, for example, a vertebrate including but not limited to mammals, reptiles, amphibians fish, and a human, from whom a target stem cell population as disclosed herein can be isolated and collected according to the methods and compositions described herein. Optionally, a subject can receive a transplantation (e.g., the target stem cell population can be implanted into a subject), for example, for the treatment, including prophylactic treatment, of a disease. For treatment of disease states which are specific for a specific animal such as a human subject, the term “subject” refers to that specific animal. The terms “nonhuman animals” and “non-human mammals” are used interchangeably herein, and include mammals such as rats, mice, rabbits, sheep, cats, dogs, cows, pigs, and non-human primates. Exemplary mammals include, but are not limited to a human, or other mammals such as a domesticated mammal, e.g., dog, cat, horse, and the like, or a production mammal, e.g., cow, sheep, pig, and the like.

The terms “substantially free” or “essentially free” are used herein to refer to considerably or significantly free of, or more than about 90%, 91%, 92%, 93%, 94%, 95%, 96%, 97%, 98% free of, or more than about 99% free of unwanted (non-target) cells.

The term “substantially pure,” with respect to a particular target stem cell population, refers to a population of cells that is at least about 75%, preferably at least about 85%, more preferably at least about 90%, and most preferably at least about 95% pure, with respect to the cells making up a total stem cell population.

The terms “therapeutic amount”, “effective amount”, or “therapeutically effective amount” are used herein interchangeably to refer to an amount of a composition of the invention sufficient to provide the intended benefit of treatment, i.e., that eliminates, reduces, or prevents the progression of a particular disease manifestation in a percentage of a population. The ordinary meaning of the term “dose” is the quantity of a composition prescribed to be taken at one time. The term “dosage” as used herein refers to dose and frequency of administration. Dosage levels are based on a variety of factors, including the type of disease, disorder, condition or injury, the age, weight, sex, medical condition of the patient, the severity of the condition, the route of administration, and the particular active agent employed. Thus the dosage regimen may vary widely, but can be determined routinely by a surgeon using standard methods. An example of a commonly used therapeutic component is the ED50, which describes the dose in a particular dosage that is therapeutically effective for a particular disease manifestation in 50% of a population.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “undesired cells or components” as used herein refers to cells or components other than mesenchymal progenitor cells (MPCs) of a cell surface antigenic profile CD34(−)/CD133(−)/CD45(−)/CD73(+)/CD90(+)/CD105(+)/CD44(−).

The term “susceptible” as used herein refers to a member of a population at risk.

### I. Adult Mesenchymal Progenitor Cells (MPCs)

According to one aspect, the described invention provides a composition comprising an isolated population of mesenchymal stem/progenitor cells (MSPCs) of a surface antigenic profile CD34(−)/CD133(−)/CD45(−)/CD73(+)/CD90(+)/CD105(+)/CD44(−). The isolated population of mesenchymal stem/progenitor cells (MSPCs) of the described characteristics can be used in the present invention regardless of the source of the population of cells.

According to one embodiment, the mesenchymal stem/progenitor cell (MSPC) population of a cell surface antigenic profile CD34(−)/CD133(−)/CD45(−)/CD73(+)/CD90(+)/CD105(+)/CD44(−) are purified from cellular components of a bone marrow aspirate acquired from a subject.

According to another embodiment, the mesenchymal stem/progenitor cell (MSPC) population of a cell surface antigenic profile CD34(−)/CD133(−)/CD45(−)/CD73(+)/CD90(+)/CD105(+)/CD44(−) is purified from peripheral blood.

According to another embodiment, the mesenchymal stem/progenitor cells (MSPCs) are capable of being mobilized by a stem cell mobilizing agent from the bone marrow into the peripheral blood. According to another embodiment, the stem cell mobilizing agent is at least one of Granulocyte-Macrophage-Colony Stimulating Factor (GM-CSF), Granulocyte-Colony Stimulating Factor (G-CSF) and Plerixafor (AMD3100).

According to another embodiment, the mesenchymal stem/progenitor cell (MSPC) population having a cell surface antigenic profile of CD34(−)/CD133(−)/CD45(−)/CD73(+)/CD90(+)/CD105(+)/CD44(−) is purified from umbilical cord blood.

According to one embodiment, the isolated population of mesenchymal stem/progenitor cells (MSPCs) is substantially free of other cell types. According to another embodiment, at least 90% of the cells in the isolated population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 95% of the cells in the isolated population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 96% of cells in the isolated population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 97% of the cells in the isolated population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 98% of the cells in the isolated population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 99% of the cells in the isolated population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 99.5% of the cells in the isolated population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 99.9% of the cells in the isolated population of mesenchymal stem/progenitor cells (MSPCs) are of the cell surface antigenic profile CD34(−)/CD133(−)/CD45(−)/CD73(+)/CD90(+)/CD105(+)/CD44(−).

According to another embodiment, the mesenchymal stem/progenitor cells (MSPCs) are considerably smaller than most mesenchymal stem cells (MSC) prepared by the conventional method. For example, the size of the mesenchymal stem/progenitor cells (MSPCs) of the described invention is 5-10 μm.

According to another embodiment, the mesenchymal stem/progenitor cells (MSPCs) are multipotent stem cells that have a capacity to differentiate into a variety of cell types including, but not limited to ectodermal cells, mesodermal cells, and endodermal cells.

According to another embodiment, the mesenchymal stem/progenitor cells (MSPCs) can form a three-dimensional spheroid when plated on a ultra low attachment plate.

According to another embodiment, the ectodermal cells differentiated from the mesenchymal stem/progenitor cells (MSPCs) comprise neurons of a peripheral and central nervous system. According to another embodiment, the neurons, which are differentiated from the mesenchymal stem/progenitor cells (MSPCs), express a neuronal marker β-Tubulin 3 (Tuj-1).

According to another embodiment, the mesodermal cells differentiated from the MSPCs include, but are not limited to, adipocytes, chondrocytes, and osteoblasts.

According to another embodiment, the mesodermal cells differentiated from the mesenchymal stem/progenitor cells (MSPCs) of the present invention can be further induced to differentiate into ectodermal cells.

According to another embodiment, the mesenchymal stem/progenitor cells (MSPCs) population is more capable of expansion in culture than are conventionally-derived adherent mesenchymal stem cells (MSC). According to another embodiment, the mesenchymal stem/progenitor cells (MSPCs) can be expanded in culture in a chemically defined medium.

According to another embodiment, exposure of the mesenchymal stem/progenitor cells (MSPCs) of the described invention to a stem cell mobilizing agent results in appearance of two populations consisting of a CD105(+)/CD44(+) population and a CD105(+)/CD44(−) population. Exemplary stem cell mobilizing agents include, but are not limited to, G-CSF, GM-CSF, and plerixafor (AMD3100).

According to some embodiments, the composition of the present invention may be formulated with an excipient, carrier or vehicle including, but not limited to, a solvent. The terms “excipient”, “carrier”, or “vehicle” as used herein refers to carrier materials suitable for formulation and administration of the composition comprising the isolated population of mesenchymal stem/progenitor cells (MSPCs) described herein. Carriers and vehicles useful herein include any such materials known in the art which are nontoxic and do not interact with other components. As used herein the phrase “pharmaceutically acceptable carrier” refers to any substantially non-toxic carrier useable for formulation and administration of the composition of the present invention in which the isolated population of mesenchymal stem/progenitor cells (MSPCs) of the present invention will remain stable and bioavailable.

The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition. For example, the pharmaceutically acceptable carrier can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.). Other suitable pharmaceutically acceptable carriers for the compositions of the present invention include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like. Such carrier solutions also can contain buffers, diluents and other suitable additives. The term “buffer” as used herein refers to a solution or liquid whose chemical makeup neutralizes acids or bases without a significant change in pH. Examples of buffers envisioned by the present invention include, but are not limited to, Dulbecco's phosphate buffered saline (PBS), Ringer's solution, 5% dextrose in water (D5W), normal/physiologic saline (0.9% NaCl). According to some embodiments, the infusion solution is isotonic to subject tissues. According to some embodiments, the infusion solution is hypertonic to subject tissues. Compositions of the present invention that are for parenteral administration can include pharmaceutically acceptable carriers such as sterile aqueous solutions, non-aqueous solutions in common solvents such as alcohols, or solutions in a liquid oil base.

According to some embodiments, the carrier of the composition of the present invention may include a release agent such as sustained release or delayed release carrier. In such embodiments, the carrier can be any material capable of sustained or delayed release of the active to provide a more efficient administration, e.g., resulting in less frequent and/or decreased dosage of the composition, improve ease of handling, and extend or delay effects on diseases, disorders, conditions, syndromes, and the like, being treated, prevented or promoted. Non-limiting examples of such carriers include liposomes, microsponges, microspheres, or microcapsules of natural and synthetic polymers and the like. Liposomes may be formed from a variety of phospholipids such as cholesterol, stearylamines or phosphatidylcholines.

The compositions of the present invention may be administered parenterally in the form of a sterile injectable aqueous or oleaginous suspension. The term “parenteral” or “parenterally” as used herein refers to introduction into the body by way of an injection (i.e., administration by injection), including, but not limited to, infusion techniques. For example, the composition of the present invention comprising an isolated population of mesenchymal stem/progenitor cells (MSPCs) can be delivered to the subject by means of a catheter adapted for delivery of the fluid compositions (i.e., compositions capable of flow) into a selected anatomical structure. According to some embodiments, parenteral administration includes but is not limited to intravascular delivery (meaning into a blood vessel), intravenous delivery (meaning into a vein), intra-arterial delivery (meaning into an artery), intraosseous delivery (meaning into the bone marrow), intramuscular delivery (meaning into a muscle), subcutaneous delivery (meaning under the skin), cardiac delivery (meaning into the heart, myocardium), etc.

The sterile composition of the present invention may be a sterile solution or suspension in a nontoxic parenterally acceptable diluent or solvent. A solution generally is considered as a homogeneous mixture of two or more substances; it is frequently, though not necessarily, a liquid. In a solution, the molecules of the solute (or dissolved substance) are uniformly distributed among those of the solvent. A suspension is a dispersion (mixture) in which a finely-divided species is combined with another species, with the former being so finely divided and mixed that it doesn't rapidly settle out. In everyday life, the most common suspensions are those of solids in liquid water. Among the acceptable vehicles and solvents that may be employed are water, Ringer's solution, and isotonic sodium chloride (saline) solution. According to some embodiments, hypertonic solutions are employed. In addition, sterile, fixed oils are conventionally employed as a solvent or suspending medium. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran.

Additional compositions of the present invention can be readily prepared using technology which is known in the art such as described in Remington's Pharmaceutical Sciences, 18th or 19th editions, published by the Mack Publishing Company of Easton, Pa., which is incorporated herein by reference.

According to another embodiment, the mesenchymal stem/progenitor cells (MSPCs) of the described invention can be used for treating a degenerative tissue condition related to musculoskeletal defects, including, but not limited to, degenerative bone disorders, osteoarthritis, and neurological injury and cardiovascular disorders.

The amount of the stem cell component in the compositions of the present invention that will be effective in the treatment of a particular disorder or condition will depend on the nature of the disorder or condition, and can be determined by standard clinical techniques. (See, for example, Goodman and Gilman's THE PHARMACOLOGICAL BASIS OF THERAPEUTICS, Joel G. Harman, Lee E. Limbird, Eds.; McGraw Hill, New York, 2001; THE PHYSICIAN'S DESK REFERENCE, Medical Economics Company, Inc., Oradell, N.J., 1995; and DRUG FACTS AND COMPARISONS, FACTS AND COMPARISONS, INC., St. Louis, Mo., 1993). The precise dose to be employed in the formulations of the present invention also will depend on the route of administration and the seriousness of the disease or disorder, and should be decided according to the judgment of the practitioner and each subject's circumstances. It is envisioned that subjects may benefit from multiple administrations of the pharmaceutical composition of the present invention.

According to some embodiments, the isolated mesenchymal stem/progenitor cells (MSPCs) having a surface antigenic profile of CD34(−)/CD133(−)/CD45(−)/CD73(+)/CD90(+)/CD105(+)/CD44(−) has at least one activity selected from the group consisting of a neuronal differentiation activity, a chondrogenic differentiation activity, an osteogenic differentiation activity, or an adipogenic differentiation activity. According to one embodiment, the isolated mesenchymal stem/progenitor cells (MSPCs) having a surface antigenic profile of CD34(−)/CD133(−)/CD45(−)/CD73(+)/CD90(+)/CD105(+)/CD44(−) has a neuronal differentiation activity. According to another embodiment, the isolated mesenchymal stem/progenitor cells (MSPCs) having a surface antigenic profile of CD34(−)/CD133(−)/CD45(−)/CD73(+)/CD90(+)/CD105(+)/CD44(−) has a chondrogenic differentiation activity. According to another embodiment, the isolated mesenchymal stem/progenitor cells (MSPCs) having a surface antigenic profile of CD34(−)/CD133(−)/CD45(−)/CD73(+)/CD90(+)/CD105(+)/CD44(−) has an osteogenic differentiation activity. According to another embodiment, the isolated mesenchymal stem/progenitor cells (MSPCs) having a surface antigenic profile of CD34(−)/CD133(−)/CD45(−)/CD73(+)/CD90(+)/CD105(+)/CD44(−) has an adipogenic differentiation activity.

According to some embodiments, the compositions according to the present invention contain at least 0.5×10³ mesenchymal stem/progenitor cells (MSPCs) having a surface antigenic profile of CD34(−)/CD133(−)/CD45(−)/CD73(+)/CD90(+)/CD105(+)/CD44(−) having at least one activity per dosage unit, selected from the group consisting of neuronal differentiation activity, chondrogenic differentiation activity, osteogenic differentiation activity, or adipogenic differentiation activity. According to some embodiments, the compositions according to the present invention contain at least 0.5×10⁴ mesenchymal stem/progenitor cells (MSPCs) having a surface antigenic profile of CD34(−)/CD133(−)/CD45(−)/CD73(+)/CD90(+)/CD105(+)/CD44(−) having at least one activity per dosage unit, selected from the group consisting of neuronal differentiation activity, chondrogenic differentiation activity, osteogenic differentiation activity, or adipogenic differentiation activity. According to some embodiments, the compositions according to the present invention contain at least 0.5×10⁵ mesenchymal stem/progenitor cells (MSPCs) having a surface antigenic profile of CD34(−)/CD133(−)/CD45(−)/CD73(+)/CD90(+)/CD105(+)/CD44(−) having at least one activity per dosage unit, selected from the group consisting of neuronal differentiation activity, chondrogenic differentiation activity, osteogenic differentiation activity, or adipogenic differentiation activity. According to some embodiments, the compositions according to the present invention contain at least 0.5×10⁶ mesenchymal stem/progenitor cells (MSPCs) having a surface antigenic profile of CD34(−)/CD133(−)/CD45(−)/CD73(+)/CD90(+)/CD105(+)/CD44(−) having at least one activity per dosage unit, selected from the group consisting of neuronal differentiation activity, chondrogenic differentiation activity, osteogenic differentiation activity, or adipogenic differentiation activity. According to some embodiments, the compositions according to the present invention contain at least 0.5×10⁷ mesenchymal stem/progenitor cells (MSPCs) having a surface antigenic profile of CD34(−)/CD133(−)/CD45(−)/CD73(+)/CD90(+)/CD105(+)/CD44(−) having at least one activity per dosage unit, selected from the group consisting of neuronal differentiation activity, chondrogenic differentiation activity, osteogenic differentiation activity, or adipogenic differentiation activity. According to some embodiments, the compositions according to the present invention contain at least 0.5×10⁸ mesenchymal stem/progenitor cells (MSPCs) having a surface antigenic profile of CD34(−)/CD133(−)/CD45(−)/CD73(+)/CD90(+)/CD105(+)/CD44(−) having at least one activity per dosage unit, selected from the group consisting of neuronal differentiation activity, chondrogenic differentiation activity, osteogenic differentiation activity, or adipogenic differentiation activity.

According to another embodiment, the compositions of the present invention can be administered by a combination therapy, whereby the pharmaceutical compositions further include one or more compatible active ingredients which are aimed at providing the composition with another pharmaceutical effect in addition to that provided by the isolated mesenchymal stem/progenitor cells (MSPCs) of the present invention.

### II. Process for Isolating and Purifying Mesenchymal Stem/Progenitor Cells (MSPCs)

According to another aspect, the described invention provides a method for isolating and purifying a population of mesenchymal stem/progenitor cells (MSPCs) of a cell surface antigenic profile CD34(−)/CD133(−)/CD45(−)/CD73(+)/CD90(+)/CD105(+)/CD44(−), wherein the method is based on cluster of differentiation (CD) molecules on a surface of a pure initial population of cells, wherein the method comprises:

(a) isolating a source of cells from a mammal;

(b) depleting CD34-positive and CD133-positive cells from (a) to obtain a first purified cell population of a cell surface antigenic profile CD34(−)/CD133(−);

(c) fractionating the first purified cell population of (b) using antibodies against cell surface antigens CD45, CD73, and CD90 to obtain a second purified cell population of the cell surface antigenic profile CD34(−)/CD133(−)/CD45 (−)/CD73(+)/CD90(+); and

(d) further fractionating the second purified cell population in (c) using antibodies against cell surface antigens CD105 and CD44 to obtain a third purified cell population of the cell surface antigenic profile CD34(−)/CD133(−)/CD45(−)/CD73(+)/CD90(+)/CD105(+)/CD44(−);

wherein the method does not employ adherent culture of an unfractionated mononuclear population.

According to one embodiment, the mesenchymal stem/progenitor cell (MSPC) population of a cell surface antigenic profile CD34(−)/CD133(−)/CD45(−)/CD73(+)/CD90(+)/CD105(+)/CD44(−) are purified from cellular components of a bone marrow aspirate acquired from a subject.

According to another embodiment, the mesenchymal stem/progenitor cell (MSPC) population of a cell surface antigenic profile CD34(−)/CD133(−)/CD45(−)/CD73(+)/CD90(+)/CD105(+)/CD44(−) is purified from peripheral blood.

According to another embodiment, the mesenchymal stem/progenitor cell (MSPC) population of a cell surface antigenic profile CD34(−)/CD133(−)/CD45(−)/CD73(+)/CD90(+)/CD105(+)/CD44(−) is purified from umbilical cord blood.

According to one embodiment of the method, depletion of the CD34-positive and the CD133-positive cells in (b) is carried out by using a magnetic bead selection system.

According to another embodiment, steps (c) and (d) are carried out by using fluorescence-activated cell sorting (FACS).

According to another embodiment, the method further comprises (e) cryopreserving the third purified cell population by admixing the third purified cell population with a cryoprotectant and storing the population at a low temperature.

According to some embodiments, the source of mesenchymal stem/progenitor cells (MSPCs) is selected from the group consisting of a bone marrow aspirate, a peripheral blood sample, or an umbilical cord or portion thereof. According to one embodiment, the source of mesenchymal stem/progenitor cells (MSPCs) is a bone marrow aspirate. According to another embodiment, the source of mesenchymal stem/progenitor cells (MSPCs) is a peripheral blood sample. According to another embodiment, the source of mesenchymal stem/progenitor cells (MSPCs) is an umbilical cord or portion thereof

According to another embodiment, the source of mesenchymal stem/progenitor cells (MSPCs) is a mobilized source sample following exposure to a stem cell mobilizing agent. According to another embodiment, the mesenchymal stem/progenitor cells (MSPCs) are capable of being mobilized by a stem cell mobilizing agent from the bone marrow into the peripheral blood. According to another embodiment, the stem cell mobilizing agent is at least one of GM-CSF, G-CSF and plerixafor (AMD3100).

According to another embodiment, the population of mesenchymal stem/progenitor cell (MSPC) isolated according to the method is substantially free of other cell types. According to another embodiment, at least 90% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 95% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 96% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 97% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 98% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 99% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 99.5% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 99.9% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−).

According to another aspect, the described invention provides a method for obtaining an enriched population of mesenchymal stem/progenitor cells (MSPCs) from a source using a size-based elutriation technique, wherein the size-based elutriation technique comprises flowing the source sample obtained from a subject through a series of increasing flow rates in an elutriation device, wherein each flow rate in the series of increasing flow rates collects a different population of cells in each flow rate fraction. According to some embodiments, the source of mesenchymal stem/progenitor cells (MSPCs) is selected from the group consisting of a bone marrow aspirate, a peripheral blood sample, or an umbilical cord or a portion thereof. According to one embodiment, the source of mesenchymal stem/progenitor cells (MSPCs) is a bone marrow aspirate. According to another embodiment, the source of mesenchymal stem/progenitor cells (MSPCs) is a peripheral blood sample. According to another embodiment, the source of mesenchymal stem/progenitor cells (MSPCs) is an umbilical cord or a portion thereof.

According to one embodiment, wherein the source of mesenchymal stem/progenitor cells (MSPCs) is a bone marrow sample, the method comprises:

(1) flowing the bone marrow sample at a first flow rate,

wherein the first flow rate allows a first flow rate fraction comprising bone marrow cells that are smaller than the mesenchymal progenitor cell population (MPCs) in the bone marrow sample to flow through the elutriation device and to be collected in a first cell collection bag of the elutriation device.

(2) increasing the first flow rate to a second flow rate,

wherein the second flow rate allows a second flow rate fraction comprising mesenchymal stem/progenitor cells (MSPCs) or bone marrow cells having a same size as the size of the mesenchymal stem/progenitor cells (MSPCs) in the bone marrow sample to flow through the elutriation device and to be collected in a second cell collection bag of the elutriation device, and

wherein the second flow rate fraction collected in the second cell collection bag comprises the enriched population of the mesenchymal stem/progenitor cells (MSPCs);

(3) recirculating the bone marrow sample comprising non-retained cells through the elutriation apparatus; and

(4) optionally increasing the second flow rate to a third flow rate, wherein the third flow rate allows a third flow rate fraction comprising bone marrow cells that are larger than the mesenchymal stem/progenitor cells (MSPCs) in the bone marrow sample to flow through the elutriation device and to be collected in a third cell collection bag of the elutriation device.

According to one embodiment, method for obtaining an enriched population of mesenchymal stem/progenitor cells (MSPCs) further comprises:

(5) depleting CD34-positive and CD133-positive cells from the collected mesenchymal stem/progenitor cells (MSPCs) of (4) to obtain a first purified cell population of a cell surface antigenic profile CD34(−)/CD133(−);

(6) fractionating the first purified cell population of (5) using antibodies against cell surface antigens CD45, CD73, and CD90 to obtain a second purified cell population of the cell surface antigenic profile CD34(−)/CD133(−)/CD45(−)/CD73(+)/CD90(+); and

(7) further fractionating the second purified cell population in (6) using antibodies against cell surface antigens CD105 and CD44 to obtain a third purified cell population of the cell surface antigenic pro file CD34(−)/CD133(−)/CD45(−)/CD73(+)/CD90(+)/CD105(+)/CD44(−);

wherein the method does not employ adherent culture of an unfractionated mononuclear population.

According to one embodiment of the method, the first flow rate ranges from about 20 ml/minute to about 40 ml/minute, and the first flow rate fraction comprises a substantial number of platelets.

According to another embodiment, the first flow rate is 25 ml/minute and the first flow rate fraction comprises a substantial number of platelets. According to another embodiment, the first flow rate is 30 ml/minute and the first flow rate fraction comprises a substantial number of platelets. According to another embodiment, the first flow rate is 35 ml/minute and the first flow rate fraction comprises a substantial number of platelets. According to another embodiment, the first flow rate is 40 ml/minute and the first flow rate fraction comprises a substantial number of platelets.

According to another embodiment, the second flow rate to obtain the enriched population of the mesenchymal stem/progenitor cells (MSPCs) ranges from about 50 ml/minute to about 90 ml/minute.

According to another embodiment, the second flow rate to obtain the enriched population of the mesenchymal stem/progenitor cells (MSPCs) is 50 ml/minute. According to another embodiment, the second flow rate to obtain the enriched population of the mesenchymal stem/progenitor cells (MSPCs) is 55 ml/minute. According to another embodiment, the second flow rate to obtain the enriched population of the mesenchymal stem/progenitor cells (MSPCs) is 60 ml/minute. According to another embodiment, the second flow rate to obtain the enriched population of the mesenchymal stem/progenitor cells (MSPCs) is 65 ml/minute. According to another embodiment, the second flow rate to obtain the enriched population of the mesenchymal stem/progenitor cells (MSPCs) is 70 ml/minute. According to another embodiment, the second flow rate to obtain the enriched population of the mesenchymal stem/progenitor cells (MSPCs) is 75 ml/minute. According to another embodiment, the second flow rate to obtain the enriched population of the mesenchymal stem/progenitor cells (MSPCs) is 80 ml/minute. According to another embodiment, the second flow rate to obtain the enriched population of the mesenchymal stem/progenitor cells (MSPCs) is 85 ml/minute. According to another embodiment, the second flow rate to obtain the enriched population of the mesenchymal stem/progenitor cells (MSPCs) is 90 ml/minute.

According to another embodiment, the third flow rate to obtain the third flow rate fraction comprising the bone marrow cells larger than the mesenchymal stem/progenitor cells (MSPCs) is greater than 90 ml/minute.

According to another embodiment, the third flow rate is greater than 90 ml/minute but less than 105 ml/minute. According to another embodiment, the third flow rate that is greater than 90 ml/minute but less than 105 ml/minute collects a population of cells comprising Hematopoietic Stem Cells (HSCs).

The first, second, or third flow rates are calculated as discussed in Table 1 in U.S. Pat. No. 6,022,306, which is incorporated herein by reference. According to some embodiments, the centrifugal elutriation separates cell particles having different sedimentation velocities, as disclosed in U.S. Pat. No. 7,201,848, which is incorporated herein in its entirety by reference. Stoke's law describes sedimentation velocity (SV) of a spherical particle, as follows:

$$SV = \frac{2}{9} \frac{{r}^{2} ( {ρ}_{p} - {ρ}_{m} )}{η} g ,$$

where, r is the radius of the particle; ρₚ is the density of the particle; ρₘ is the density of the liquid medium; η is the viscosity of the medium; and g is the gravitational or centrifugal acceleration. Because the radius of a particle is raised to the second power in the Stoke's equation and the density of the particle is not, the size of a cell, rather than its density, greatly influences its sedimentation rate. This explains why, if the particles have similar densities, larger particles generally remain in a chamber during centrifugal elutriation, while smaller particles are released.

According to one embodiment of the method, depletion of the CD34-positive and the CD133-positive cells in (5) is carried out by a magnetic bead selection system.

According to another embodiment, steps (6) and (7) are carried out by fluorescence-activated cell sorting (FACS).

According to another embodiment, the method further comprises removing undesired cells or components., i.e., cells other than those of the cell surface antigenic profile CD34(−)/CD133(−)/CD45(−)/CD73(+)/CD90(+)/CD105(+)/CD44(−).

According to another embodiment, the method further comprises cryopreserving the enriched population of the mesenchymal stem/progenitor cells (MSPCs) by admixing the third purified cell population with a cryoprotectant and storing the population at a low temperature.

According to another embodiment, the enriched population of the mesenchymal stem/progenitor cells (MSPCs) obtained according to the claimed method is substantially free of other cell types. According to another embodiment, at least 90% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 95% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 96% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 97% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 98% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 99% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 99.5% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−). According to another embodiment, at least 99.9% of the cells in the population of mesenchymal stem/progenitor cells (MSPCs) are of the cell surface antigenic profile CD34(−)/CD133(−)/CD45(−)/CD73(+)/CD90(+)/CD105(+)/CD44(−).

According to another embodiment, the source of mesenchymal stem/progenitor cells (MSPCs) is a source sample mobilized upon exposure to a stem cell mobilizing agent. According to another embodiment, the stem cell mobilizing agent is at least one of GM-CSF, G-CSF and plerixafor (AMD3100).

Exemplary elutriation devices for use in the present invention include, but are not limited to those described in WO 2011/069117, the entire contents of which are incorporated herein by reference. For example, a selected source sample comprising a mesenchymal stem/progenitor cell (MSPC) population is introduced into a generally funnel-shaped separation chamber located on a spinning centrifuge. A flow of liquid elutriation buffer or low density liquid is then introduced into the chamber containing the peripheral blood sample. As the flow rate of the liquid elutriation buffer solution is increased through the chamber (e.g., in a stepwise manner), the liquid sweeps smaller sized, slower-sedimenting cells toward an elutriation boundary within the chamber, while larger, faster-sedimenting cells migrate to an area of the chamber where the centrifugal force and the sedimentation (drag) forces are balanced. Accordingly, as the source sample comprises many different populations of stem cells, elutriation results in the separation of these different stem cell populations present in source sample into distinct populations, where smaller stem cells are fractionated from stem cells which are larger in size. Exemplary elutriation devices include, but are not limited to, commercially available elutriation devices, for example, the ELUTRA® centrifuge manufactured by Gambro BCT, Inc. The ELUTRA CELL SEPARATION SYSTEM® enables the separation of cell populations into multiple fractions based on both size and density, enabling cell enrichment, depletion, concentration, and washing all within a functionally-closed system, and in less than one hour enables enrichment of stem cell populations directly from leukapheresis products without antibodies or preprocessing. The ELUTRA CELL SEPARATION SYSTEM® uses counterflow centrifugal elutriation, where fluid flows through cell layers in a centrifugal field in order to separate cell populations.

According to some embodiments, when the source is a peripheral blood sample, other elutriation devices can be used, including but not limited to, the COBE® Spectra apheresis system, the TRIMA® system and the TRIMA ACCEL® system, manufactured by Gambro BTC Inc., as well as other commercially available elutriation devices used to separate blood components. According to some embodiments, the peripheral blood is fractionated using a cell separator such as the COBE® Spectra Apheresis System. The COBE® SPECTRA™ centrifuge is described in U.S. Pat. Nos. 4,425,172; 4,708,712; and 6,022,306, which are incorporated herein by reference. In such an embodiment, the peripheral blood sample is drawn into a cell separator such as the COBE® Spectra Apheresis System, and, optionally, an anticoagulant solution is added to the blood to keep it from clotting during the procedure. The blood/anticoagulant admixture cycles through a centrifuge to separate stem cell populations and mononuclear cells from the other blood components and plasma. The system pumps the separated stem cells into a collection bag for storage, while the other blood components and plasma return to the patient. All tubing sets and needles used are sterile, so there is no risk of disease transmission. Other blood-separation apparatuses, such as the apparatus described in U.S. Pat. No. 5,722,926, issued Mar. 3, 1998; U.S. Pat. No. 5,951,877, issued Sep. 14, 1999; U.S. Pat. No. 6,053,856, issued Apr. 25, 2000; U.S. Pat. No. 6,334,842, issued Jan. 1, 2002; U.S. patent application Ser. No. 10/884,877 filed Jul. 1, 2004; U.S. Pat. No. 7,201,848; U.S. Pat. No. 6,022,306; U.S. Pat. No. 6,589,526; U.S. Patent Applications 2008/0035585; US2008/0318756 and 2009/0104626 can be used. The entire disclosure of each of these U.S. patents and patent applications is incorporated herein by reference.

According to some embodiments, the isolated population of mesenchymal stem/progenitor cells (MSPCs) can be cryopreserved (e.g., frozen) in a cryopreservation medium and stored for long periods of time, being capable of use on thawing, for example, for use in therapeutic purposes such as regenerative therapy or medicine. According to one embodiment, the cryopreservation medium comprises 10% DMSO, 50% FCS, and 40% RPMI-1640 medium. Methods of cryopreservation of stem cells are known by those of ordinary skill in the art and are disclosed in U.S. Patent Applications 2008/0220520, 2009/0022693, 2008/0241113 and 2005/0106554 and U.S. Pat. Nos. 5,759,764 and 7,112,576 and 7,604,930, which are incorporated herein in their entirety by reference.

For cryopreservation, a population of mesenchymal stem/progenitor cells (MSPCs), isolated by the methods as disclosed herein can be suspended in a balanced salt solution, e.g. Dulbecco's Phosphate Buffered Saline (DPBS) and may be placed on ice for at least about 15 minutes in preparation for cryopreservation. The preparation may comprise adding cryopreservation media to the target stem cell population or to a substantially pure population of target stem cells and then subjecting the mixture to several temperature reduction steps to reduce the temperature of the population to a final temperature of about −90° C., utilizing a controlled rate freezer or other suitable freezer system (dump-freeze monitored or a freeze container (Nalgene)). Suitable control rate freezers include, but are not limited to, Cryomed Thermo Form a Controlled Rate Freezer 7454 (Thermo Electron, Corp.), Planar Controlled Rate Freezer Kryo 10/16 (TS Scientific), Gordinier, Bio-Cool—FTS Systems, and Asymptote EF600, BIOSTOR CBS 2100 series.

Cryopreservation media may be prepared comprising media and DMSO. About 3 ml of DPBS may be added to a container, such as, for example, a 50 ml conical tube. About 1 ml of human serum albumin (HSA) may be added to the about 3 ml of DPBS and then chilled for about ten minutes on ice. About 1 ml of the chilled 99% DMSO is added to the HSA and DPBS to prepare the final cryopreservation media. Cryopreservation media and the cell population may then be placed on ice for about 15 minutes before the cryopreservation media is added to the cell sample. Batch processing may be used for aliquoting cryopreservation media into a cell sample. For example, a single aliquot of about 100 of the target stem cell population, or a substantially pure population of target stem cells, may be combined with about 3 ml of DPBS, 1 nil of HSA, and about 1 ml of 99% DMSO. About 2 aliquots of about 200 μl of MSC cell suspension may be combined with about 6 nil of DPBS, 2 ml of HSA, and about 2 ml of 99% DMSO. About 5 aliquots of cell sample may be combined with about 15 ml of DPBS, about 5 ml of HSA, and about 5 ml of 99% DMSO. About 10 aliquots of cell sample may be combined with about 30 ml of DPBS, about 10 ml of HSA, and about 10 nil of 99% DMSO.

Other cryopreservation media may be used. For example, cryopreservation media with cryopreservation agents may be used to maintain a high cell viability outcome post-thaw, such as, for example, CryoStor CSIO or CS5 (Biolife), embryonic cryopreservation media supplemented with propanediol and sucrose (Vitrolife), or SAGE media (Cooper Surgical). Glycerol may be used with other cryopreservation agents, such as, DMSO, or may be used alone at a concentration of about 10% in a media with suitable protein.

III. Method for Differentiating Mesenchymal Progenitor Cells (MPCs) into Neuronal Cells

According to another aspect, the described invention provides a method for differentiating a population of mesenchymal stem/progenitor cells (MSPCs) into neuronal cells, comprising:

(a) culturing a population of mesenchymal progenitor cells (MPCs) of an antigenic profile CD34(−)/CD133(−)/CD45(−)/CD73(−)/CD90(+)/CD105(+)/CD44(−) in a chemically defined growth medium devoid of an animal serum;

(b) generating a spheroid by plating the population of mesenchymal progenitor cells (MPCs) on a low-attachment plate;

(c) plating and culturing the population of mesenchymal progenitor cells (MPCs) from the spheroid in (b) in a defined medium for neuronal differentiation;

(d) obtaining a neuronal cell population differentiated from the population of mesenchymal stem/progenitor cells (MSPCs).

According to one embodiment of the method, the neuron-like cells express a neuronal marker β-Tubulin 3 (Tuj-1). According to another embodiment of the method, culturing in (c) is continued for at least 21 days.

Unless defined otherwise, all technical and scientific terms used herein have the same meaning as commonly understood by one of ordinary skill in the art to which this invention belongs. Although any methods and materials similar or equivalent to those described herein also can be used in the practice or testing of the described invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

It must be noted that as used herein and in the appended claims, the singular forms “a”, “and”, and “the” include plural references unless the context clearly dictates otherwise. All technical and scientific terms used herein have the same meaning

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

The described invention may be embodied in other specific forms without departing from the spirit or essential attributes thereof and, accordingly, reference should be made to the appended claims, rather than to the foregoing specification, as indicating the scope of the invention.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Isolation of CD34(−)/CD133(−)/CD44(−)/CD45(−)/CD73(+)/CD90(+)/CD105(+) Mesenchymal Stem Cells (MSC)

A morphologically and phenotypically distinct population of mesenchymal stem cells (MSC), which lacks expression of the classic MSC marker CD44, and which exhibits a physical size that is more equivalent to HSCs than the traditional MSCs, has been identified using magnetic cell depletion and polychromatic flow cytometry combined with elutriation.

Sample Processing

Fresh, unprocessed bone marrow (BM) was obtained from healthy donors (Lonza, MD). Samples were processed under aseptic conditions. BM was diluted 1:1 in DPBS (without Ca²⁺ and Mg²⁺), followed by RBC lysis using Pharm Lyse 1× lysis buffer (BD Pharmingen). After RBC lysis, cells were washed with 0.5% human serum albumin (HSA) in DPBS and centrifuged at 680 g for 15 minutes at 4° C. Next, cells were counted for viability and resuspended in 0.5% HSA/DPBS and processed for cell isolation. Fresh, mobilized leukapheresis products were purchased from AllCells, LLC (Emeryville, Calif.) or collected from healthy volunteers at NeoStem Laboratory in Cambridge Mass. under an IRB approved protocol. Three days prior to apheresis, healthy donors received daily subcutaneous injections of G-CSF (Granulocyte-Colony Stimulating Factor—Neupogen®, Amgen Inc., Thousand Oaks, Calif., 480 μg/day). A certified staff technician conducted the collection of the apheresis product over the course of 2 to 3 hours. After the collection of the mobilized apheresis product, cells were diluted to a final concentration of 2.5×10⁸/mL in 300 mL of 0.5% HSA/PBS prior to elutriation as described below.

Magnetic Activated Cell Sorting (MACS™)

Bone marrow isolated from a human adult was depleted of CD34-positive and CD133-positive cells by Miltenyi® magnetic bead selection. Specifically, after determining cell viability of the lysed BM, CD34 and CD133 expressing cells were depleted using MACS™ CD34 and CD133 microbead kits (Miltenyi Biotech) according to manufacturer's instructions. After 30 minute incubation, cells were washed with 0.5% HSA/DPBS and centrifuged at 680 g for 15 minutes and the cell pellet then resupended. Cell depletion was performed with the MACS™ LS column and QuadroMACS™ separator (Miltenyi Biotech) according to the manufacturer's instructions. Both the enriched and depleted fractions were examined for cell viability, cell number and cell size distribution using a cellometer analyzer (Nexelcom).

Fluorescence Activated Cell Sorting (FACS)

The depleted fraction was subjected to a stringent polychromatic flow cytometric sorting strategy. A panel of antibodies representing the most common markers for plastic adherent BM MSCs was used for their prospective identification. This 6-color panel, which includes antibodies to CD45, CD73, CD90, CD105 and CD44 and the cell viability marker, 7-Aminoactinomycin D (7-AAD; a fluorescent nucleic acid dye) was used for fluorescence-activated cell sorting (FACS) to analyze CD34/CD133-depleted BM for populations that contain MSC activity.

For example, the CD34/CD133-depleted fractions were re-suspended in FACS staining buffer (R&D Systems) and incubated with the following antibodies: CD45-pacific blue (PB; Beckman Coulter), CD73-allophycocyanin (APC; BD Biosciences), CD90-fluorescein isothiocyanate (FITC; BD Biosciences), CD105-phycoerythrin (PE; BD Biosciences) and CD44-allophycocyanin H7 (APC-H7; BD Biosciences) on ice for 30 minutes. Following staining, cells were washed with DPBS, centrifuged at 680 g for 10 minutes, re-suspended in buffer, and passed through a 40 μM filter (BD Biosciences). The viability dye 7-Aminoactinomycin D (7-AAD; Beckman Coulter) was added prior to sorting.

A principle of flow cytometry data analysis is to selectively visualize the cells of interest while eliminating results from unwanted particles e.g. dead cells and debris. This procedure is called gating. Cells can been gated according to physical characteristics, as well as fluorescence-based properties. For instance, subcellular debris and clumps can be distinguished from single cells by size, estimated by forward scatter. Also, dead cells have lower forward scatter and higher side scatter than living cells. In addition, the gating strategy can be employed to delineate subpopulations of cells expressing specific cell-surface markers based on characteristic fluorescence parameters of the markers.

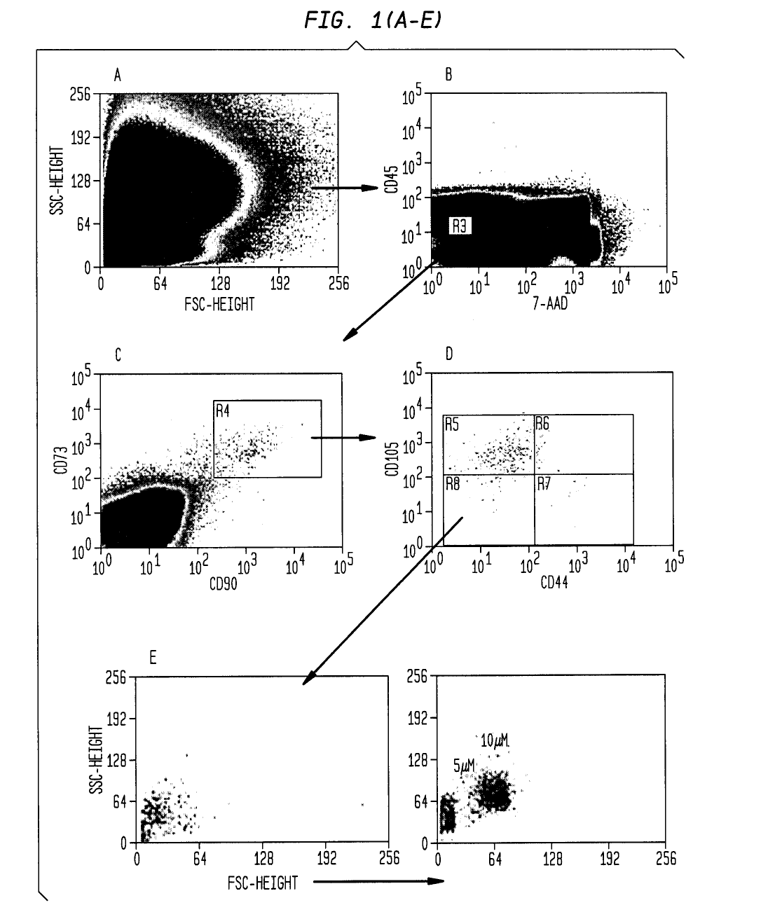

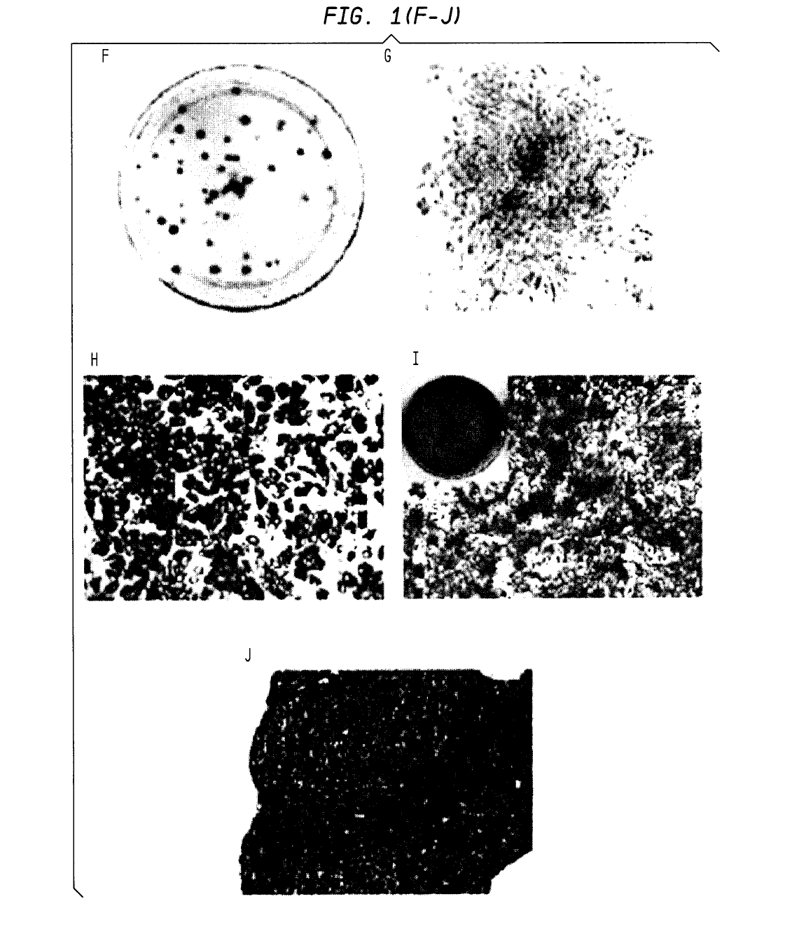

Cell sorting was carried out with a Beckman Coulter high speed Moflo XDP cell sorter (Beckman Coulter). The Moflo XDP was equipped with four lasers (488 nm, 642 nm 405 nm, and 355 nm). Forward scatter (FSC) threshold was set low to ensure inclusion of small cells. FIG. 1A shows mononuclear cells (MNCs) initially displayed on a Side Scatter (SSC) vs. Forward Scatter (FSC) color density plot of BM cells.

Cells were analyzed and sorted using a sequential gating strategy. An initial gate was set on CD45 versus 7AAD (gate R3). Therefore, first, live cells were isolated based on the exclusion of 7-Aminoactinomycin D (7-AAD; a fluorescent nucleic acid dye), and subsequently, the cell population, which is negative for the pan-hematopoietic marker CD45, was identified. (CD45⁻/7-AAD⁻ cells; FIG. 1B). Next, the CD45⁻ live (7-AAD) cells were then displayed on a CD73 versus CD90 plot, and then a second gate was drawn to include a rare population of CD73⁺/CD90⁺ cells, identified as candidate MSCs. (FIG. 1C, gate R4). Following this, CD45⁻CD73⁺CD90⁺ (7-AAD) viable cells were further applied on a third plot of CD105 versus CD44 and with quadrant gates delineated for CD105⁺ or CD44⁺ cells in order to identify CD105⁺/CD44⁻ subset. The isolated CD34(−)/CD133(−)/CD45(−)/CD73(+)/CD90(+)/CD105(+) cells were sorted again to collect cells that lack expression of the classic homing receptor CD44. The subgating showed that a large majority of the cells were CD105⁺ but CD44⁻. (FIG. 1D, gate R5). Populations of the following 4 subpopulations (if any) were sorted directly to tubes containing ice-cold (4° C.) chemically defined, serum-free culture medium (MSCGM-CD, Lonza): CD45⁻/CD73⁺/CD90⁺/CD105⁺/CD44⁻; CD45⁻/CD73⁺/CD90⁺/CD105⁺/CD44⁺; CD45⁻/CD73⁺/CD90⁺/CD105⁻/CD44⁻; CD45⁻/CD73⁺/CD90⁺/CD105⁻/CD44⁺. Therefore, the MSCs isolated according to the described invention exhibit a cell surface antigenic profile distinct from conventional MSCs, which express CD44, widely regarded as a classic MSC marker. (Sackstein, R. et al., “Ex vivo glycan engineering of CD44 programs human multipotent mesenchymal stromal cell trafficking to bone,” Nat. Med., 14: 181-187 (2008)).

Confirming results first described in the priority U.S. Provisional Application No., U.S. 61/554,290, filed Nov. 1, 2011, Qian et al. have independently described MSPCs from bone marrow that lack expression of CD44 (Qian, H. et al., “Primary mesenchymal stem and progenitor cells from bone marrow lack expression of CD44 protein,” J. Biol. Chem., 287(31): 25795-25807 (published online May 31, 2012)). Similar to the instant invention, Qian et al. also found that the CD44⁻ cells acquire CD44 expression during culture, indicating that adherence and growth on plastic alters the in vivo antigenic profile of MSPCs. Such observations underscore that markers found on culture-expanded MSCs may not be present on their precursor cells in vivo. However, a distinguishing phenotype in the present study is that CD105 was found to be a positive marker for the prospective isolation of CD44⁻ MSPCs, which contrasts with the report from Qian et al., where CD44⁻ cells had diminished CD105 expression, but did express CD271. In the present study, the inventors found that following expansion, CD44− MSPCs do not express CD271. There are conflicting reports in the art describing the regulation of CD271 expression with expansion. For example, some reports suggest that CD271 is downregulated following expansion, in agreement with results described in the present study (Jones, E. A. et al., “Isolation and characterization of bone marrow multipotential mesenchymal progenitor cells,” Arthritis Rheum., 46: 3349-3360 (2002); Jones, E. et al., “Large-scale extraction and characterization of CD271+ multipotential stromal cells from trabecular bone in health and osteoarthritis: implications for bone regeneration strategies based on uncultured or minimally cultured multipotential stromal cells,” Arthritis Rheum., 62: 1944-1954 (2010)). Other reports do not implicate such regulation. (Battula, V. L. et al., “Isolation of functionally distinct mesenchymal stem cell subsets using antibodies against CD56, CD271, and mesenchymal stem cell antigen-1,” Haematologica, 94: 173-184 (2009)).

CD44 is a multifunctional class I integral transmembrane glycoprotein that is expressed on a number of cell populations in the BM. (Ponta, H. et al., “CD44: from adhesion molecules to signalling regulators,” Nat. Rev. Mol. Cell Biol., 4: 33-45 (2003)). When a back gating strategy was used in the present study, a CD45⁻ cell population that was CD44⁺ could be identified. These cells were CD73⁺ but lacked CD90, a marker used to satisfy a stringent MSC criterion. (Dominici, M. et al., “Minimal criteria for defining multipotent mesenchymal stromal cells. The International Society for Cellular Therapy position statement,” Cytotherapy, 8: 315-317 (2006)). Using elutriation/FACS these rare CD45⁻CD90⁻CD73⁺ cells could be fractionated into subpopulations of CD105⁺CD44⁻ and CD105⁻CD44⁺ cells. However, only the CD105⁻CD44⁺ subpopulation adhered and grew in culture. In contrast to CD45⁻CD73⁺CD105⁺CD44⁻ cells, these CD44⁺ cells grew slowly as a spheroid over a prolonged period in culture, could be dispersed non-enzymatically for expansion, and showed mesenchymal differentiation potential. This is in contrast to the Qian et al. study (Qian, H. et al., “Primary mesenchymal stem and progenitor cells from bone marrow lack expression of CD44 protein,” J. Biol. Chem., 287(31): 25795-25807 (published online May 31, 2012)), where the CD44⁺ cells were not expandable in culture.

Maintenance and Expansion of FACS-Sorted CD45⁻CD73⁺CD105⁺CD44⁻ Cells

The FACS-sorted CD45⁻CD73⁺CD105⁺CD44⁻ cells were plated onto plastic in chemically defined, serum free medium, mesenchymal stem cell growth medium-chemically defined (MSCGM-CD). The sorted cells were centrifuged at 680 g for 15 minutes at 4° C., resuspended in MSCGM-CD, and seeded into either 6-well or 10 cm dishes. Cultures were maintained in a humidified incubator with 5% CO₂ and low oxygen (5% O₂) at 37° C. The cells were left untouched for 5 days, and on day 6 non-adherent cells were aspirated off, and fresh MSCGM-CD media then was added. Following this, adherent cultures were maintained by changing the media twice weekly. The cultures were continuously fed for 10-14 days until they reached 70-80% confluence.

Cells were expanded following subculturing and used for differentiation assays and flow cytometric analysis as described below. Unstained cells and isotype negative control samples were used to set photomultiplier tube (PMT) voltage for baseline fluorescence and to set quadrant statistics for analyzing positive fluorescence above baseline. Compensation was manually adjusted using known positive single color stained samples together with an unstained control. Data acquisition and analysis was performed using Summit Software (Beckman Coulter). A minimum of 500,000 events was recorded as a list mode file for further analysis.

Size Characterization of FACS-sorted CD45−/CD73+/CD90+/CD105+/CD44− cells

Cells from the population of CD45⁻/CD73⁺/CD90⁺/CD105⁺/CD44⁻ were back-gated and displayed on a side scatter/forward scatter (SSC/FSC) colour density plot of FIG. 1A to reveal their location. (FIG. 1E, left plot). Backgating of the CD45⁻/CD73⁺/CD90⁺/CD105⁺/CD44⁻ cells onto the SSC/FSC density plot revealed the location of these rare cells in a region near the lymphocyte population, i.e., populations representing small cell size. Standardized flow cytometric beads were used to confirm their size. Microbeads of standard size demonstrate that the CD44⁻ cells were between 5 and 12 microns (FIG. 1E, right plot).

CFU-F Analysis of FACS-Sorted CD45−/CD73+/CD90+/CD105+/CD44− cells

According to one embodiment, to examine the CFU-F potential of the sorted fractions, cells were plated at a density of 1 cell/cm² in a 6 well dish in mesenchymal stem cell growth medium-chemically defined (MSCGM-CD). Cells were grown for 14 days; thereafter the media were removed, cells were washed with DPBS, and fixed with methanol (BDH) for 5 minutes at room temperature. Next, the methanol was removed and cells were air dried for 5 minutes at room temperature. To stain cultures, 2 mL of Geimsa (EMD Chemicals) staining solution was added to each well and incubated for 10 minutes at room temperature. Afterwards, the staining solution was removed. Cells were washed with distilled water to remove unbound stain and further washed until the wells were clear.

MSC activity, in the form of characteristic CFU-F, was found in the fraction lacking expression of CD44, widely considered an important MSC marker. FIG. 1F shows a representative image of typical CFU-F from the sorted CD45⁻CD73⁺CD105⁺CD44⁻ cells and FIG. 1G shows a higher power image of a single colony (at 4× magnification). The CD44⁻ cells rapidly proliferated and formed characteristic CFU-F by day 12.

Differentiation Potential of Expanded FACS-sorted CD45⁻CD73⁺CD105⁺CD44⁻ Cells

The FACS-sorted CD44− cells were expanded in culture. Following expansion, these cells were able to differentiate into osteoblasts, chondrocytes and adipocytes under defined conditions in vitro (FIG. 1H-1J), indicating that they function as MSPCs. Differentiation assays were performed according to the Methods described below in Example 2 following enrichment with elutriation. FIG. 1H shows differentiated adipocytes detected using Oil Red O stain for lipids; FIG. 1I shows differentiated osteblasts detected using Alizarin Red S stain; and FIG. 1J shows differentiated chondroblasts detected using safranin-O stain. Similar results were seen in 4 other BM samples from different donors. (Data not shown).

### Example 2

### Enrichment of CD34(−)/CD133(−)/CD45(−)/CD73(+)/CD90(+)/CD105(+)/CD44(−) Mesenchymal Progenitor Cells (MSPCs)

Following isolation of the CD34(−)/CD133(−)/CD45(−)/CD73(+)/CD90(+)/CD105(+)/CD44(−) cell population, the physical size of the isolated MSCs in freshly isolated samples was analyzed. Since conventional MSCs have been defined post-cultivation from an unfractionated mononuclear population, it has not been possible to determine the physical size of MSCs. Post-cultivation MSCs, which are isolated via conventional methods using Ficoll/Plastic adherence, are large fibroblast-like cells. However, backgating of the FACS-sorted CD45⁻/CD73⁺/CD90⁺/CD105⁺/CD44⁻ cells onto the SSC/FSC density plot revealed the location of these rare cells in a region near the lymphocyte population, i.e., populations representing small cell size (FIG. 1E, left plot), and further, microbeads of standard size showed that the CD44⁻ cells were between 5 and 12 microns (FIG. 1E, right plot).

Ficoll has been used to fractionate non-homogeneous cell populations based on density, whereas elutriation fractionates cells based on size. In order to address the small size of the FACS sorted CD44− MSPCs and to enrich the cells based on their small size prior to FACS, unprocessed bone marrow from healthy donor subjects was fractionated based on cell size using the Elutra® system (Caridian BCT).

The Elutra® Cell Separation system (CaridianBCT) uses counter-flow centrifugal elutriation (CCE) and was programmed to enrich the different cell types based primarily on size and secondarily on density to separate populations of cells into more specific cell fractions. Briefly, approximately 2-3×10⁹ nucleated cells (NC) from lysed BM were re-suspended in 100 mL of 0.5% HSA/DPBS and loaded onto the Elutra system. Lysed BM cells or apheresis products (see above for details) were fractionated under a constant centrifugation rate of 2,400 rpm, and 5 successive elutriated fractions of 450 to 900 mL were collected in 0.5% HSA/DPBS using progressive increases in pump speed. Each collected fraction was centrifuged at 680 g for 20 minutes at 4° C. to pellet the cells.

Elutriation separated the bone marrow into five distinct fractions based on size: Fraction 35 (elutriated at 35 ml/min); Fraction 70 (elutriated at 70 ml/min); Fraction 90 (elutriated at 90 ml/min); Fraction 110 (elutriated at 110 ml/min); and Fraction >110 (elutriated at >110 ml/min). To examine the CFU-F potential of the elutriated fractions prior to FACS sorting, cells were plated at a density of 1.0 cell/cm² in a 6 well dish in MSCGM-CD. Cells were grown for 14 days; thereafter the media was removed, cells were washed with DPBS, and fixed with methanol (BDH) for 5 minutes at room temperature. Next, the methanol was removed and cells were air dried for 5 minutes at room temperature. To stain cultures, 2 mL of Geimsa (EMD Chemicals) staining solution was added to each well and incubated for 10 minutes at room temperature. Afterwards, the staining solution was removed and cells were washed with distilled water to remove unbound stain and further washed until wells were clear.

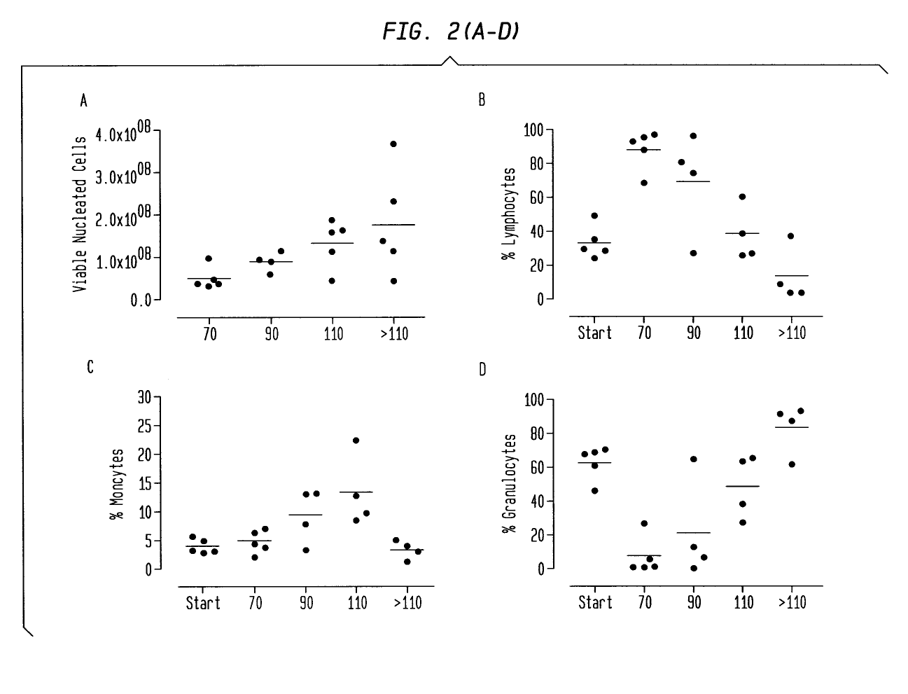

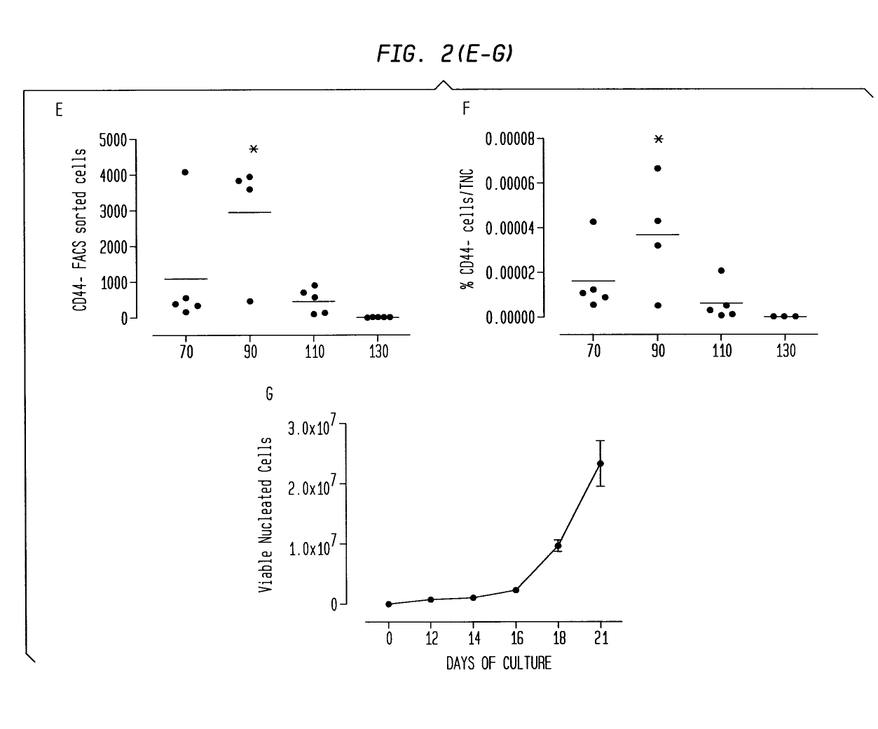

Following elutriation of the bone marrow, the different fractions were subjected to CD34/CD133 depletion and the FACS sorting strategy described in Example 1. Platelets were removed almost entirely in the first elutriated fraction (35 ml/min, Frac 35), which contained very few nucleated cells and was discarded. The cell yield and composition from all the subsequent collected fractions is shown in FIG. 2A-D. Distribution of viable nucleated cells recovered from the various fractions is shown in FIG. 2A; percentage (%) of lymphocytes in FIG. 2B; percentage (%) of monocytes in FIG. 2C; percentage (%) of granulocytes in FIG. 2D. Counterflow centrifugation elutriation (CCE) is able to separate the lymphocyte population (Fracs 70 and 90) from the granulocytes, which were collected primarily in the largest cell fractions (Fracs 110 and >110), and to a lesser extent from monocytes. The majority of nucleated cells elutriated in the later fractions, with 45% of cells found in Fr>110 alone (FIG. 2A).

Fractionation of human BM using counter flow centrifugal elutriation (CCE) combined with FACS using the 5 antibody/viability marker panel shows that CD45⁻CD73⁺CD90⁺CD105⁺CD44⁻ MSPCs elute as small cells (70 and 90 ml/min). FIG. 2E shows the quantification of FACS sorted CD44⁻ cells from the 4 fractions. Peak recovery was found in fraction 90. FIG. 2F shows the percentage (%) of rare CD45⁻CD73⁺CD90⁺CD105⁺CD44⁻ cells from the various fractions when normalized to Tenascin-C (TNC), an extracellular matrix protein, which demonstrates that fraction 90 contains the bulk of cells. CD45⁻CD73⁺CD90⁺CD105⁺CD44⁻ cells from fraction 90 were sorted and the cumulative growth curve was calculated over 21 days after initial plating calculated as day 0. (FIG. 2G).

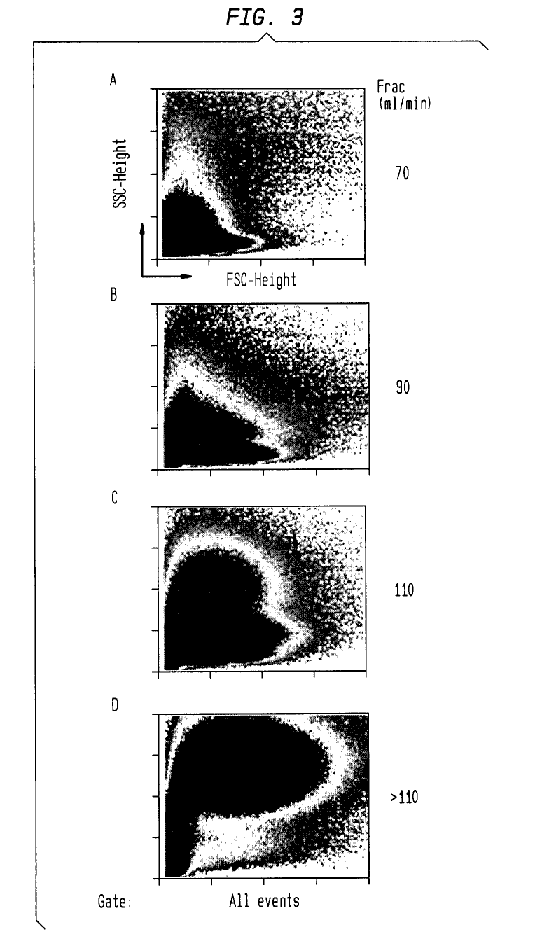

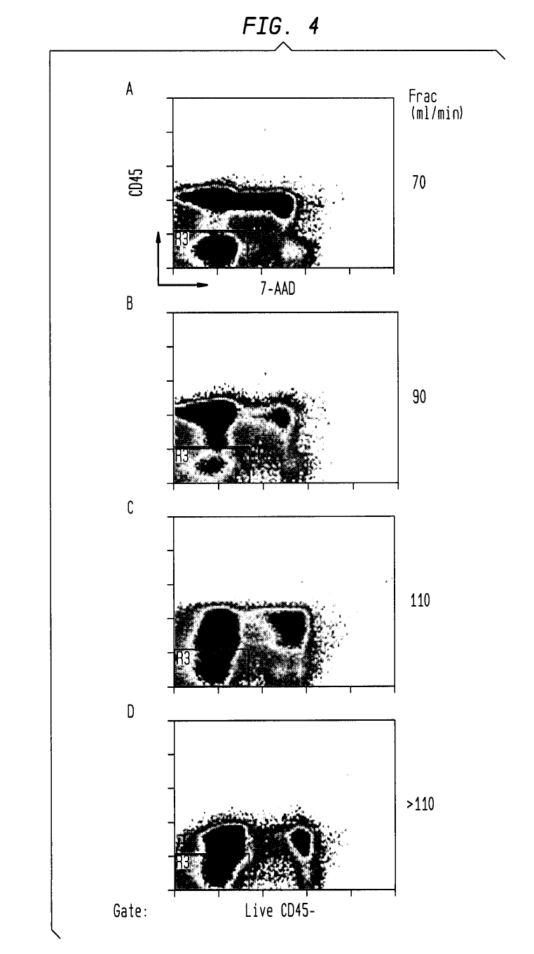

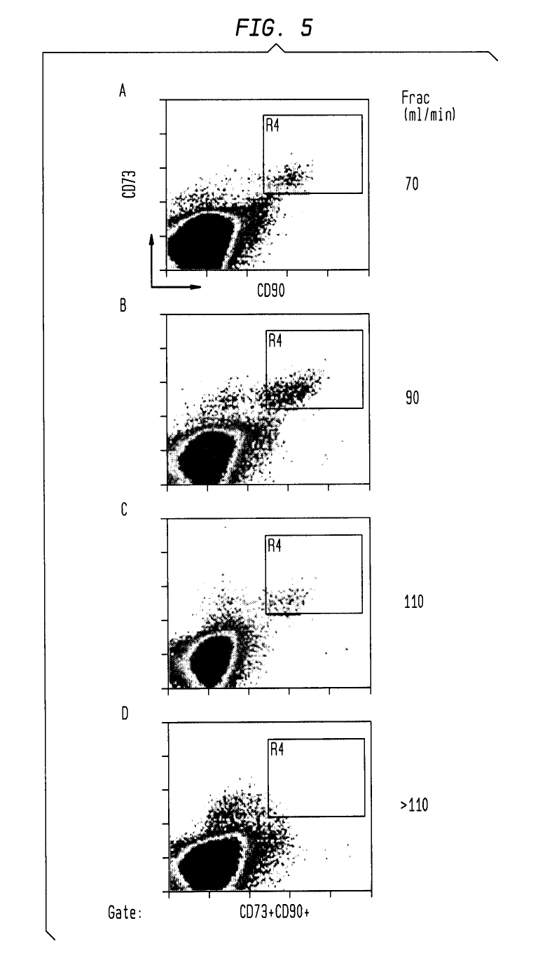

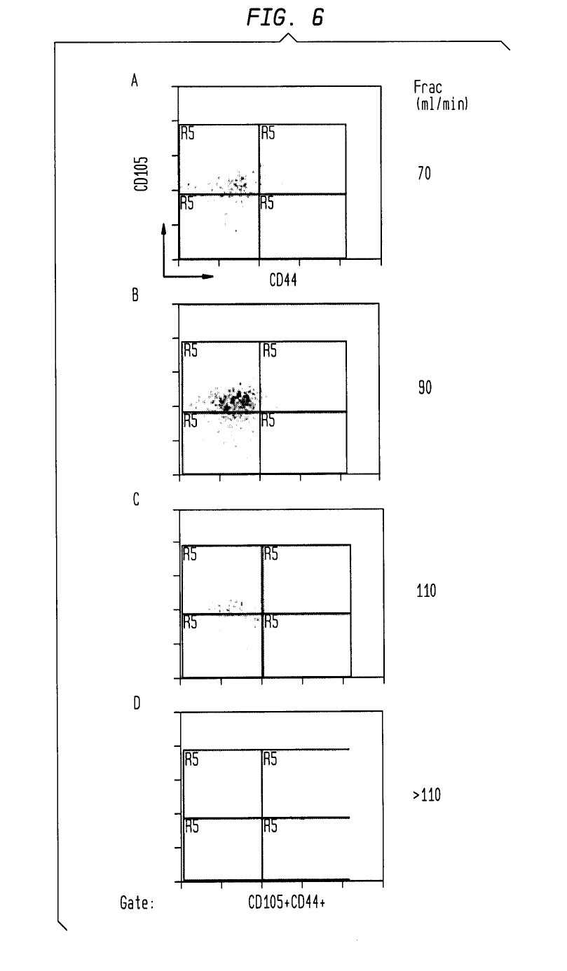

FIGS. 3-7 shows the scatter plots of a Fluorescence-Activated Cell Sorting (FACS) analysis of the four Elutra® fractions of bone marrow following depletion of CD34(+)/CD133(+) cells, staining for the hematopoietic marker CD45, and subsequent gating for CD73/CD90 and CD105/CD44 markers. The cell separation in each fraction based on size can also be seen by the change in the SSC/FSC pseudo-color density plots (FIG. 3). Representative displays of SSC/FSC color density plots of elutriated fractions 70, 90, 100-110, >110 is shown in FIGS. 3A, 3B, 3C, and 3D, respectively. For each of the elutriated fractions 70, 90, 100-110, and >110, the display of CD45/7AAD density plot was used to identify CD45⁻/7-AAD⁻ (live) cells (gate R3) (shown in FIGS. 4A, 4B, 4C, and 4D, respectively), which was subgated onto a CD73/CD90 density plot and identified a cluster of live CD45⁻CD73⁺CD90⁺ cells (gate R4) (shown in FIGS. 5A, 5B, 5C, and 5D, respectively), which was further subgated onto a CD105/CD44 antigen plot. Display of the CD105⁺CD44⁻ plot on live CD45⁻CD73⁺CD90⁺ gated cells is shown in FIGS. 6A, 6B, 6C, and 6D for fractions 70, 90, 100-110, and >110, respectively. The sort window shows a cluster of CD105⁺CD44− cells (R5).

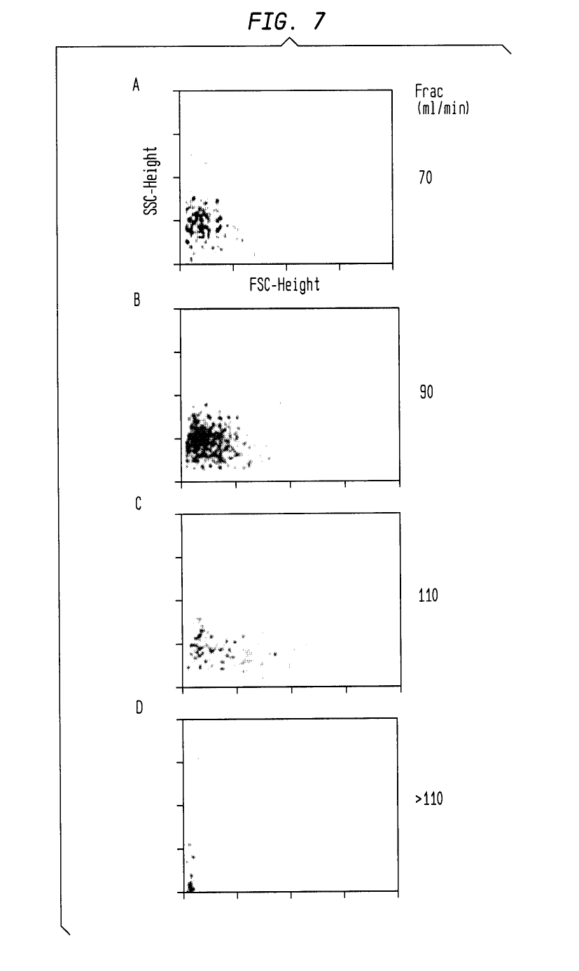

The sorted live CD45⁻CD73⁺CD90⁺CD105⁺CD44⁻ cells (gate R5) are distributed close to the location of lymphocytes when back-gated onto a SSC-Height/FSC-Height dot plot (shown in FIGS. 7A, 7B, 7C, and 7D for fractions 70, 90, 100-110, and >110, respectively).

CD45⁻CD73⁺CD90⁺CD105⁺CD44⁻ MSPCs were found in fraction 70 and peaked in fraction 90, confirming their small size (FIGS. 2E-F, 6A-B and 7A-B). The CD44⁻ MSPC population was extremely rare, with approximately 1 in 2 million events in fraction 90 being live CD45⁻CD73⁺CD90⁺CD105⁺CD44⁻ cells (FIG. 2F). Despite their rarity, sorted cells from fraction 90 underwent a 10,000-fold expansion in culture 21 days after the initial sort (FIG. 2G).

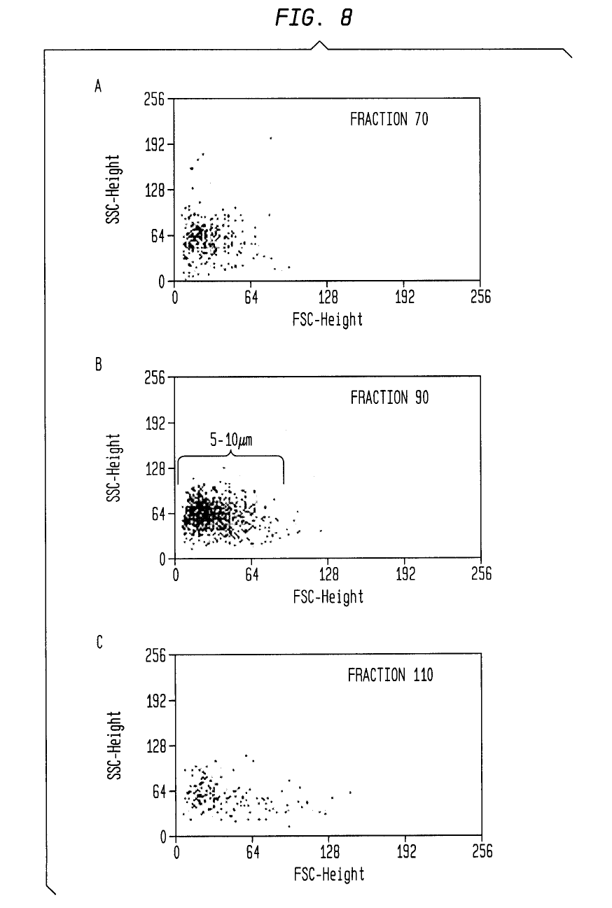

In order to verify the size of the CD34(−)/CD133(−)/CD45(−)/CD73(+)/CD90(+)/CD105(+)/CD44(−) cells, flow cytometric beads of known size were characterized based on side scatter (SSC) vs forward scatter (FSC) properties. (FIG. 8). The size of the CD34(−)/CD133(−)/CD45(−)/CD73(+)/CD90(+)/CD105(+)/CD44(−) cells were measured to be in the range of 5-10 μm, a size comparable to or slightly smaller than HSCs, which is about 10 μm.

Because of the lack of data on the physical size of MSPCs within the BM (Jones, E. et al., “Human bone marrow mesenchymal stem cells in vivo,” Rheumatology (Oxford), 47: 126-131 (2008)), it was not known where these cells would fractionate using CCE. Because MSCs were known to be large cells in vivo (Id.), it could be anticipated that MSPCs may elutriate in the later fractions, which contain contaminating monocytes and granulocytes. However, the CD44⁻ MSPCs were enriched in early fractions that also contain other stem and progenitor cell populations, such as hematopoietic stem cells (HSCs) and very small embryonic-like stem cells (VSELs) (FIGS. 3-7). VSELs represent a population of developmentally early stem cells residing in adult tissues. (Ratajczak, M. Z. et al., “Very small embryonic-like stem cells: characterization, developmental origin, and biological significance,” Exp. Hematol., 36: 742-751 (2008)). A previous study in mice similarly demonstrated that BM cells from the earliest elutriated fractions using CCE can be characterized as having a primitive potential that contributes to multiple tissues in recipient mice. (Jones, R. J. et al., “Characterization of mouse lymphohematopoietic stem cells lacking spleen colony-forming activity,” Blood. 88: 487-491 (1996); Krause, D. S. et al., “Multi-organ, multi-lineage engraftment by a single bone marrow-derived stem cell,” Cell, 105: 369-377 (2001)). Colter et al. also reported the presence of small, rapidly self-renewing cells isolated from human BM, which demonstrated robust differentiation into both, osteocytes and adipocytes. (Colter, D. C. et al., “Identification of a subpopulation of rapidly self-renewing and multipotential adult stem cells in colonies of human marrow stromal cells,” Proc. Natl. Acad. Sci. USA., 98: 7841-7845 (2001)). MSCs were isolated based on their FS^{lo}/SS^{lo} property, which enabled the enrichment of these small rapidly proliferating cells. (Smith, J. R. et al., “Isolation of a highly clonogenic and multipotential subfraction of adult stem cells from bone marrow stroma,” Stem Cells, 22: 823-831 (2004)).

### Example 3

### Expandability and Differentiation Potential of the FACS-sorted MSPCs Expandability

The CD34/CD133 depleted, CD45(−)/CD73(+)/CD90(+)/CD105(+)/CD44(−) cells are expandable in a chemically defined growth medium (e.g., MSCGM-CD, TheraPEAK™, Cat #00190632), Lonza®), which is devoid of animal proteins. After expansion, the cells can be cultured in chemically defined conditions to generate differentiated cells.

For example, the FACS-sorted CD44(−) cells are plated in a chemically defined medium that is devoid of animal serum (which contains proteins that promote adherence) and maintained in culture for 5 days without changing media. Under this condition, adherent cells are not evident until 5-8 days post culture. In contrast, according to the conventional method, which relies on seeding an impure mononuclear fraction to isolate adult BM-derived MSCs, non-adherent cells are removed and adherent cells are thought to give rise to MSCs after 72 hours. Further, their antigen expression profile is determined retrospectively using flow cytometric analysis.

Differentiation Potential

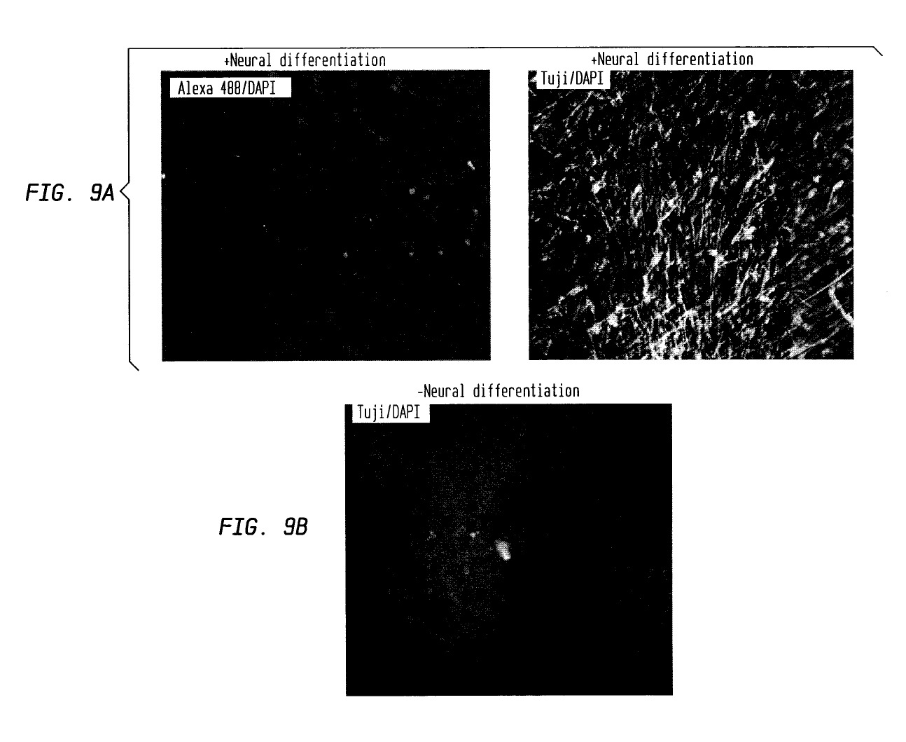

In order to examine the differentiation potential of the purified CD34(−)/CD133(−)/CD45(−)/CD73(+)/CD90(+)/CD105(+)/CD44(−) mesenchymal progenitor cells, spheroids, a solid structure of cells formed in a sphere, were generated by plating cells on ultra low-attachment plates (Costar®, ultra-low cluster plates, 6-well, Corning, Cat #3471). After the formation of spheroids, cells were plated in defined conditions for neuronal differentiation. After a 21 day period in defined neural induction/differentiation media, cells were fixed and stained for Tuj-1 (green), which represents human β-Tubulin 3, a structural protein expressed in neurons of the peripheral nervous system (PNS) and central nervous system (CNS). As shown in FIG. 9A, treatment of the CD34/CD133 depleted, CD45(−)/CD73(+)/CD90(+)/CD105(+)/CD44(−) cells with a neuronal differentiation medium resulted in a phenotypic change of the cells into a neuronal lineage as evidenced by upregulation of the neuronal marker Tuj-1. In contrast, untreated CD34(−)/CD133(−)/CD45(−)/CD73(+)/CD90(+)/CD105(+)/CD44(−) MPC cells were unreactive to Tuj-1 antibody. (FIG. 9B).

CD34(−)/CD133(−)/CD45(−)/CD73(+)/CD90(+)/CD105(+)/CD44(−) MPC cells also can be differentiated into adipocytes and osteoblasts, cell types derived from a mesodermal lineage, by treating the CD34(−)/CD133(−)/CD45(−)/CD73(+)/CD90(+)/CD105(+)/CD44(−) cells with mesodermal differentiation medium. The mesodermal cells can be further differentiated into an ectodermal cells.

Examples of mesodermal differentiation medium, include, but are not limited to, hMSC Osteogenic Differentiation Medium (Lonza Cat#PT-3002); Adipocyte Differentiation Medium comprising DMEM/Low glucose (Gibco® Cat#10567); 10% FBS (Invitrogen, Cat#12662-029); human Insulin (Sigma Cat#I2643-50MG); Indomethacin (Sigma Cat#I7378-5G); IBMX, (Sigma Cat#I7018-250MG), and Dexamethasone (Calbiochem Cat#265005); and hMSC Chondrocyte Differentiation Media (Lonza Cat#PT3003).

Data presented in this study show that a rare BM population of small, CD45⁻CD73⁺CD90⁺CD105⁺CD44⁻ cells functions as MSPCs. These MSPCs can be isolated from CD34/CD133-depleted BM by a combination of CCE and FACS sorting.

### Example 4

### Comparison of Cell Surface Antigen Expression Profiles of Isolated Elutriated/FACS-Sorted MSPCs with Conventionally Isolated MSPCs

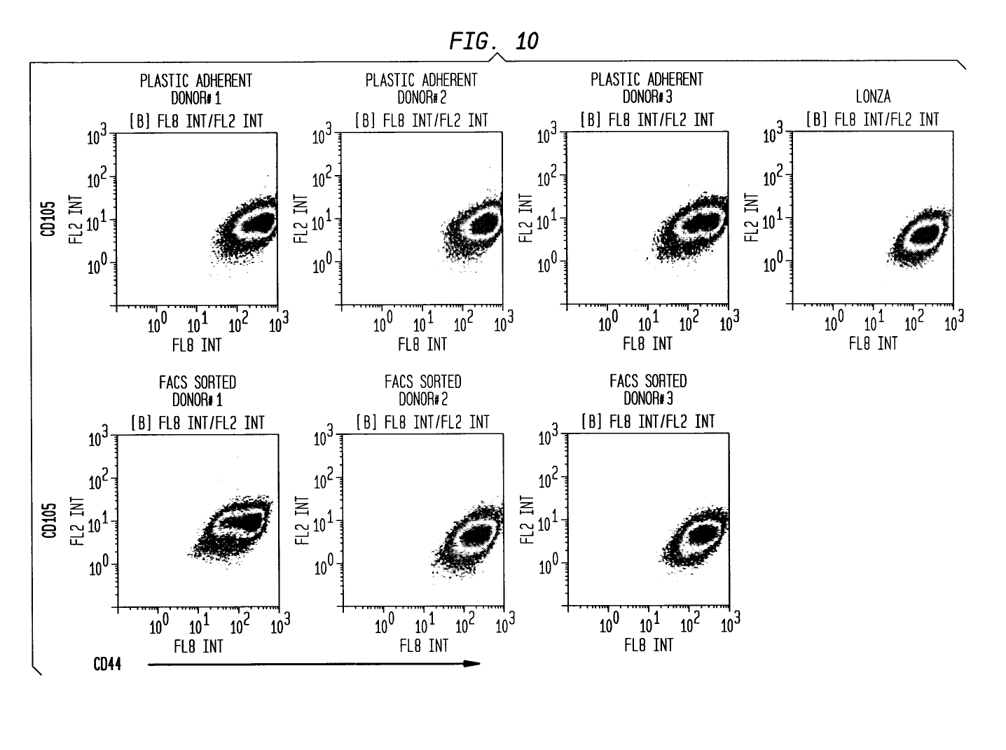

The cell surface antigen expression profile of the MSCs isolated by conventional methods (Ficoll/plastic adherence) was compared with the cell surface antigenic profile of the MPCs isolated according to the described invention. FACS-sorted MSPCs were isolated as described in Example 1. A density gradient/plastic adherence “conventional” method was also used to isolate BM-MSPCs. Mononuclear cells (MNCs) from donor matched BM were isolated by Ficoll density gradient fractionation (Ficoll-Paque Premium, 1.077, GE Healthcare). MNCs were carefully removed, washed and resuspended in chemically-defined mesenchymal stem cell growth medium (MSCGM-CD, Lonza) or culture medium composed of αMEM/Glutamax (Gibco) with 10% FBS (Invitrogen). Following enumeration of MNCs, cells were plated onto plastic dishes at a density of 1.0×10⁶ cells/cm² growth area in MSCGM-CD or αMEM/Glutamax with 10% FBS. Cultures were maintained in a humidified incubator with 5% CO₂ and low oxygen (5% O₂) at 37° C. After 72 hours, non-adherent cells were aspirated off, and the adherent cells were washed with 5 mL of pre-warmed PBS, fresh MSCGM-CD medium or αMEM/Glutamax with 10% FBS culture media was then added. Adherent cultures were maintained by changing the media twice weekly. The cultures were continuously fed for 10-14 days until they reached 70-80% confluence. Cells were expanded following subculturing and used for differentiation assays and flow cytometric analysis as described below. FIG. 10 shows flow cytometric histograms of mesenchymal progenitor cells (MPCs) isolated by a conventional method using Ficoll/plastic or by using Fluorescence-Activated Cell Sorting (FACS) according to the described invention. The isolated cells were positive for CD105/CD44 and treatment of the cells with vehicle for 3 days did not alter the expression of CD105/CD44.

This Example shows that once expanded in culture, MSPCs acquire CD44 and exhibit a similar immunophenotype to, but a more robust expansion and differentiation potential than, MSCs obtained by standard plastic adherence methods. Contrary to the widely held view that MSCs in vivo are large cells (Jones, E. et al., “Human bone marrow mesenchymal stem cells in vivo,” Rheumatology (Oxford), 47: 126-131 (2008)), and that CD44 represents a reliable and commonly cited marker for post-cultivated BM MSCs (Boxall, S. A. et al., “Markers for characterization of bone marrow multipotential stromal cells, Stem Cells Int., 2012: 975871 (2012)), this Example shows that BM contains a primitive population of small MSPCs that lack CD44, and that the acquisition of CD44 expression is a post-culture phenomenon.

### Example 5

### Comparison of Cell Surface Antigen Expression Profiles of Isolated Elutriated/FACS-Sorted MSPCs with Conventionally Isolated MSPCs Upon Expansion

Elutriated/FACS sorted CD44⁻ cells from Fraction 90 that had been cultured for 3 passages were compared to donor-matched MSCs isolated from BM by the conventional method of ficoll purification followed by plastic adherence to compare the cell surface antigen profile of elutriated/FACS sorted MSPCs with that of conventionally isolated MSPCs. Following expansion to passage 3, FACS sorted or elutriation/FACS sorted cells and donor matched MSCs were harvested using HyQtase (Hylone) and re-suspended at 10⁵ cells per 100 nt, of FACS staining buffer. Cells were incubated with antibodies (listed in Table 1 below) on ice and protected from light for 30 minutes.

TABLE 1
List of Antibodies used

| | Color | | | | |
|---|---|---|---|---|---|
| Antibody | Conjugate | Clone | Isotype | Vendor | Cat# |
| HLA-ABC | FITC | G46-2.6 | Mouse | BD | 555552 |
| | | | IgG2a,κ | Pharmingen | |
| HLA-DR | APC | G46-2 | Mouse | BD | 560896 |
| | | | IgG2a,κ | Pharmingen | |
| CD10 | APC | HI10a | Mouse | BioLegend | 312210 |
| | | | IgG1,κ | | |
| CD11b | APC | ICRF44 | Mouse | BD | 550019 |
| | | | IgG1,κ | Pharmingen | |
| CD14 | FITC | M5E2 | Mouse | BD | 555397 |
| | | | IgG2a,κ | Pharmingen | |
| CD15 | FITC | HI98 | Mouse | BD | 555401 |
| | | | IgG1,κ | Pharmingen | |
| CD19 | FITC | HIB19 | Mouse | BD | 555412 |
| | | | IgG1,κ | Pharmingen | |
| CD34 | APC | 581 | Mouse | BD | 560941 |
| | | | IgG1,κ | Pharmingen | |
| CD44 | APC-H7 | G44-26 | Mouse | BD | 560532 |
| | | | IgG2a,κ | Pharmingen | |
| CD49d | FITC | 9F10 | Mouse | BD | 560840 |
| | | | IgG1,κ | Pharmingen | |
| CD49e | APC | NKI-SAM-1 | Mouse | BioLegend | 328012 |
| | | | IgG2b,κ | | |
| CD54 | FITC | 84H10 | Mouse | Beckman | IM076U |
| | | | IgG1,κ | Coulter | |
| CD56 | PB | HCD56 | Mouse | BioLegend | 318326 |
| | | | IgG1,κ | | |
| CD61 | FITC | V1-PL2 | Mouse | BD | 555753 |
| | | | IgG1,κ | Pharmingen | |
| CD71 | FITC | M-A712 | Mouse | BD | 561938 |
| | | | IgG2a,κ | Pharmingen | |
| CD73 | APC | AD2 | Mouse | BD | 560847 |
| | | | IgG1,κ | Pharmingen | |
| CD73 | PE-Cy7 | AD2 | Mouse | BD | 561258 |
| | | | IgG1,κ | Pharmingen | |
| CD90 | FITC | 5E10 | Mouse | BD | 555595 |
| | | | IgG1,κ | Pharmingen | |
| CD90 | PE | 5E10 | Mouse | BD | 555596 |
| | | | IgG1,κ | Pharmingen | |
| CD105 | PE | 266 | Mouse | BD | 560839 |
| | | | IgG1,κ | Pharmingen | |
| CD105 | PE | SN6 | Mouse | Invitrogen | MHCD10504 |
| | | | IgG1,κ | | |
| CD106 | APC | 51-10CP | Mouse | BD | 551147 |
| | | | IgG1,κ | Pharmingen | |
| CD133 | APC | AC133 | Mouse | Miltenyi | 130-090-826 |
| | | | IgG1,κ | | |
| CD146 | PE | P1H12 | Mouse | BD | 530315 |
| | | | IgG1,κ | Pharmingen | |
| CD271 | PE | ME20.4 | Mouse | Miltenyi | 130-093-819 |
| | | | IgG1,κ | | |
| CD271 | APC | ME20.4 | Mouse | Miltenyi | 130-092-283 |
| | | | IgG1,κ | | |
| CXCR4 | APC | 12G5 | Mouse | BD | 555976 |
| | | | IgG2a,κ | Pharmingen | |
| MSCA1 | FITC | W8B2 | Mouse | Miltenyi | 130-093-587 |
| | | | IgG1,κ | | |
| MSCA1 | APC | W8B2 | Mouse | Miltenyi | 130-093-589 |
| | | | IgG1,κ | | |
| SSEA4 | AlexaFluor | MC813-70 | Mouse | BD | 560219 |
| | 647 | | IgG3,κ | Pharmingen | |
| STRO-1 | FITC | STRO1 | Mouse IgGM | BioLegend | 340105 |

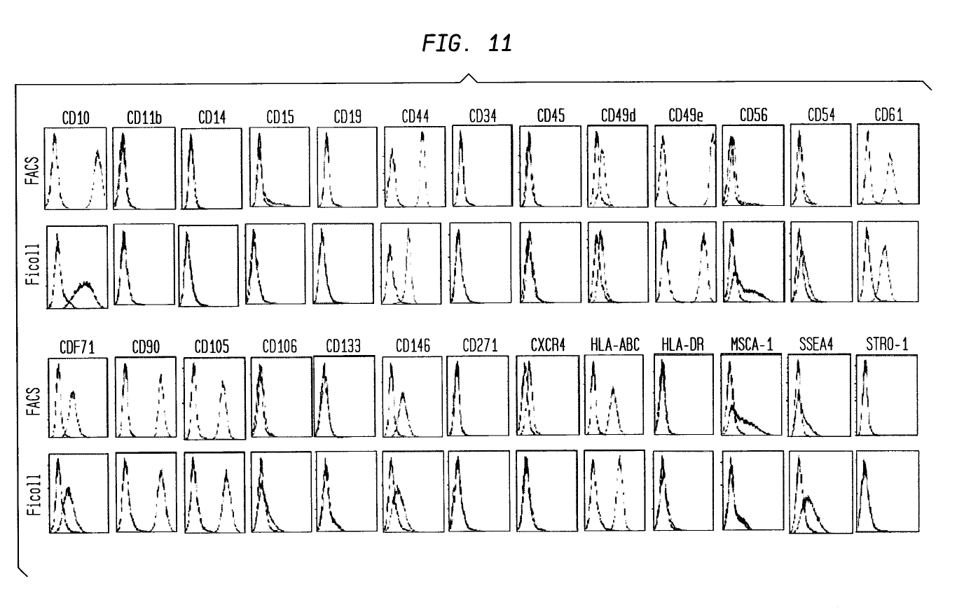

Following staining, cells were washed in buffer and resuspended in FACS staining buffer (BD Pharmingen). Cell acquisition was performed using a Galios flow cytometer (Beckman Coulter). For analysis, 30,000 events were collected and analyzed using Kaluza software (Beckman Coulter). FIG. 11 shows single colour flow cytometric analysis of selected cell surface proteins on passage 3 culture expanded CD45⁻CD73⁺CD90⁺CD105⁺CD44⁻ cells (top panels in each row, FACS) compared to passage 3 donor matched MSCs isolated using conventional methods (bottom panels in each row, Ficoll).). FIG. 11 shows that the small, CD45⁻CD73⁺CD90⁺CD105⁺CD44⁻ MSPCs acquired CD44 expression following culture on plastic. In addition, the cultured FACS-sorted cells expressed other typical markers reported for MSCs isolated by conventional methods, such as CD10, CD49d, CD49e, CD61, CD71, and CD146 (FIG. 4). Also similar to MSCs, the cultured cells from Fraction 90 were negative for CD14, CD15, CD19, CD34, CD133 and HLA-DR. Both expanded MSPCs and conventional, cultured MSCs were negative for CD271 and STRO-1, two proteins that have been proposed as markers for the prospective isolation of MSCs from BM. (Churchman, S. M. et al., “Transcriptional profile of native CD271+ multipotential stromal cells: evidence for multiple fates, with prominent osteogenic and Wnt pathway signaling activity,” Arthritis Rheum., 64(8): 2632-2643 (2012); Gronthos, S. et al., “Molecular and cellular characterisation of highly purified stromal stem cells derived from human bone marrow,” J. Cell Sci., 116: 1827-1835 (2003); Cox, G. et al., “High abundance of CD271(+) multipotential stromal cells (MSCs) in intramedullary cavities of long bones,” Bone, 50: 510-517 (2012)). There were some differences between cells prepared by the two methods, notably in CD10, MSCA-1, CXCR4, CD56 and CD54, with MSCA-1 being the most prominent; however, the functional consequences of these differences are not clear.

Effect of a Mobilizing Agent on Cell Surface Antigen Profile of Isolated Elutriated/FACS-Sorted MSPCs and Conventionally Isolated MSPCs Upon Expansion

To examine the effects of G-CSF treatment on the cell surface phenotype of elutirated/FACS sorted cells versus BM MSCs isolated using “conventional” methods, cells were treated with 10 ng/mL of recombinant human G-CSF (R&D Systems) for 4 consecutive days in serum-free αMEM/Glutamax medium. Following treatment, cells were harvested using HyQTase, counted, and resuspended in 100 μL of staining buffer with CD105-PE and CD44-APC-H7 for 15 minutes on ice. The expression of CD44 cell surface antigen was assessed using Flow cytometric analysis performed using a Gallios flow cytometer (Beckman Coulter) and 30,000 events were collected and analyzed using Kaluzza software (Beckman Coulter).

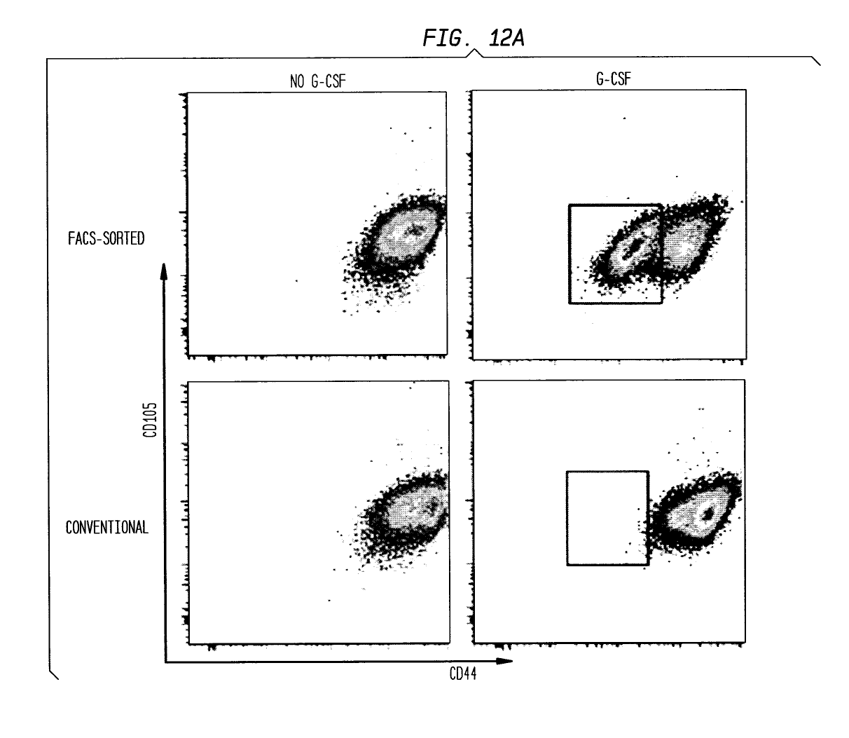

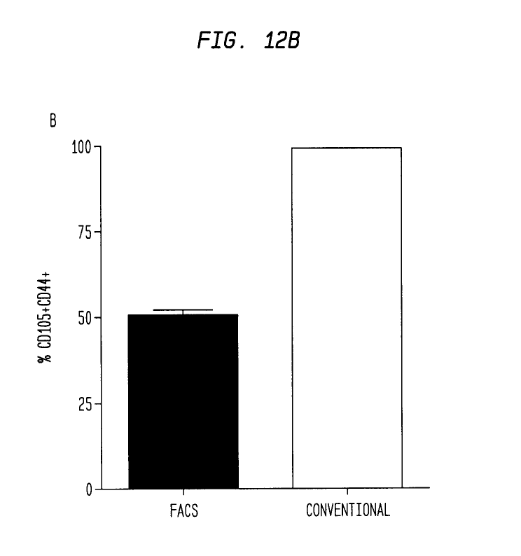

Despite the inability to detect these rare cells within mobilized blood, BM-derived CD44⁻ cells in culture do respond to G-CSF. When treated with recombinant human (rh) G-CSF (10 ng/mL) for 4 consecutive days, CD44 expression from FACS sorted MSPCs was diminished. Exposure of the MSC population isolated using FACS to G-CSF resulted in the appearance of two distinct populations of cells as indicated by flow cytometry, i.e., a CD105(+)CD44(+) cell population and CD105(+)CD44(−) cell population. (FIG. 12A-B). In comparison, MSCs isolated from the BM using the conventional method did not show diminished CD44 expression in response to rhG-CSF treatment (FIG. 12A-B).

Recent evidence indicates that MSCs play a supportive role in maintaining HSCs in a specialized niche within the BM. (Bianco, P., “Minireview: The stem cell next door: skeletal and hematopoietic stem cell “niches” in bone,” Endocrinology, 152: 2957-2962 (2011)). This raises the question of whether MSCs, along with HSCs, may be mobilized by G-CSF from the BM to the peripheral circulation. The concept that MSCs mobilize and circulate remains controversial with a lack of supportive evidence. (Jones, E. et al., “Human bone marrow mesenchymal stem cells in vivo,” Rheumatology (Oxford), 47: 126-131 (2008); Boxall, S. A. et al., “Markers for characterization of bone marrow multipotential stromal cells,” Stem Cells Int., 2012: 975871 (2012)). BM MSCs respond to G-CSF by promoting the transmigration of CD34+ cells in culture. (Ponte, A. L. et al., “Granulocyte-Colony-Stimulating Factor Stimulation of Bone Marrow Mesenchymal Stromal Cells Promotes CD34+ Cell Migration Via a Matrix Metalloproteinase-2-Dependent Mechanism,” Stem Cells Dev., PMID 22651889 (2012)). Mobilized peripheral blood products obtained from healthy donors following a 3 day course of G-CSF were thus evaluated for the presence of rare CD44⁻ cells using our method of either FACS or elutriation/FACS. However, rare CD45⁻CD73⁺CD90⁺CD105⁺CD44⁻ cells were not detected (data not shown). However, using the described method, rare CD45⁻CD73⁺CD90⁺CD105⁺CD44⁻ cells could not be detected in G-CSF mobilized peripheral blood products obtained from healthy donors (data not shown). Nevertheless, culture expanded CD44⁻ cells do respond to G-CSF, since following G-CSF exposure, their acquired CD44 expression, is partially lost (FIG. 12). Whether CD44⁻ MSPCs mobilize in response to other combinations of cytokines and growth factors remains to be determined. (Hoggatt, J. et al., “Many mechanisms mediating mobilization: an alliterative review,” Curr. Opin. Hematol., 18: 231-238 (2011)).

### Example 6

### Comparison of Expandability and Differentiation Potential of the Elutriated/FACS Sorted MSPCs with Those of Conventionally Isolated MSCs

Differentiation assays were performed comparing elutriated/FACS sorted CD44⁻ cells from fraction 90 to donor matched BM MSCs prepared by ficoll purification and plastic adherence following expansion of FACS or elutriated/FACS sorted cells and donor matched control MSCs obtained from lysed BM. Both preparations were expanded in culture until passage 3.

For adipogenic differentiation, cells were plated at a density of 1.0×10⁵ cells/well in a 6 well dish in MSCGM-CD medium and placed in a humidified chamber with 5% CO₂, regular oxygen tension (21% O₂) at 37° C. After 24 hours, the cells were washed with PBS and 2 mL of adipogenic maintenance media formulated with DMEM/low glucose (Gibco), 10 μg/mL human insulin (Invitrogen), 10% FBS (Invitrogen), 1% Pen-Strep (Invitrogen). After 3 days, the media were changed to adipogenic induction media formulated with DMEM/low glucose, 10 μg/mL human insulin, 100 μM indomethacin (Invitrogen), 0.5 mM IBMX (Invitrogen), 1 μM dexamethasone (Calbiochem). After an additional two cycles of maintenance-induction media changes, cells were incubated with adipogenic maintenance media for an additional week. After this, cells were fixed with 10% formalin (Sigma) and stained with Oil Red O (Sigma). To quantify adipogenesis, 1 mL of 70% isopropanol was added to each well; plates were placed in an incubator at 37° C. for 30 minutes to extract Oil Red O. The supernatant was removed and extracted Oil Red O was measured spectrophotometrically at 510 nm.

For chondrogenic differentiation, 5.0×10⁵ cells contained in 100 μL of MSCGM-CD (Lonza) were added to a 6.5 mm transwell permeable support insert (0.4 μM, polycarbonate membrane, Corning). This insert was placed into a well of a 24 well plate and centrifuged at 150 g for 5 minutes. Afterwards, 500 μL of chondrogenic maintenance media (Lonza) was added to the bottom well and 100 μL of fresh chondrogenic media was added to the top well of the pelleted cells and placed in a humidified chamber at 5% CO₂, regular oxygen tension (21% O₂) at 37° C. After 24 hours, 10 ng/mL of TGF-β3 (Lonza) was added to the media (chondrogenic induction media) to induce chondrogenesis. Fresh chondrogenic induction media changes were made three times weekly. After 21 days, the cell pellets were fixed in 10% formalin and prepared for paraffin embedding and 5 μM slices were stained with safranin 0.

For osteogenic differentiation, cells were plated at 4000 cells/cm² per well in a 6 well dish. After 24 hours, the media was changed to osteogenic induction medium consisting of αMEM/GLUTAMAX, 10% FBS, 1% Pen-strep, 50 μM ascorbic acid (Sigma), 10 mM β-glycerophosphate (Sigma), and 100 nM dexamethasone. Half media changes were made twice weekly. After 21 days, cells were fixed with 10% formalin and stained with 40 mM Alizarin Red S (Sigma) (pH=4.2).

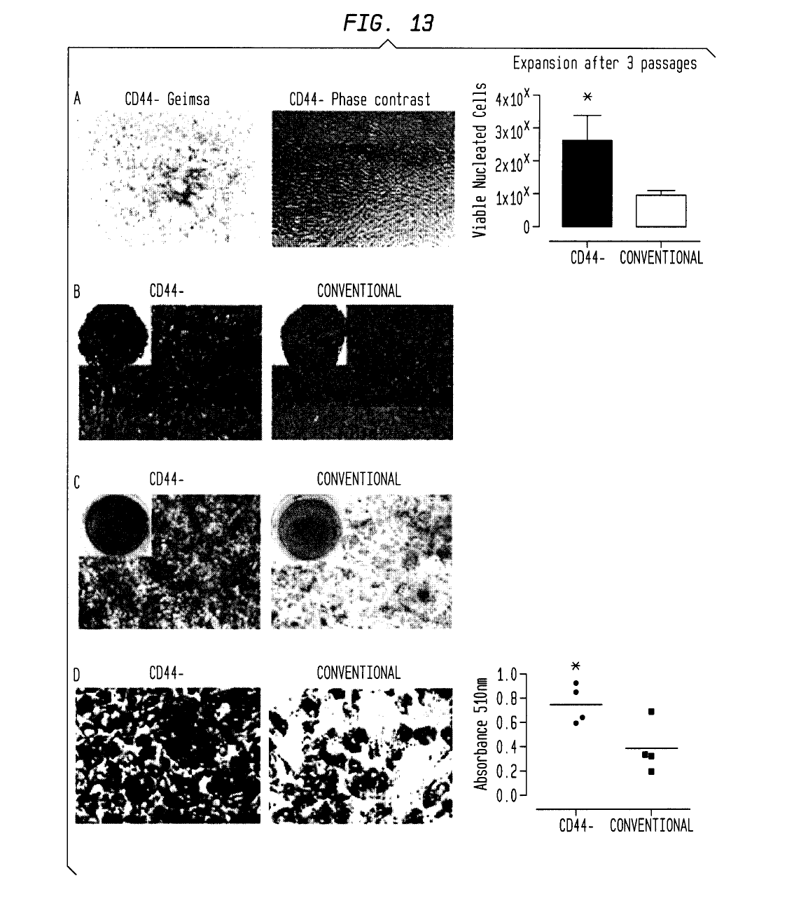

Representative colony appearance from elutriated/FACS-sorted cells on day 12 is shown in FIG. 13A (left panel), as well as cell morphology after passage 1 (FIG. 13A, center panel). Furthermore, elutriated/FACS sorted CD44⁻ cells demonstrated a greater expansion capacity over 3 passages compared to BM MSCs isolated using the conventional method (FIG. 6A, right panel, P<0.041). Following this, cells were used for the various differentiation assays in defined culture conditions in vitro.

After 21 days in osteogenic medium, sorted CD44⁻ cells stained more densely with Alizarin Red S compared to conventional MSCs, indicating an enhanced mineralization in culture (FIG. 13B). In addition, pellets of CD44⁻ cells expanded in chondrogenic induction medium for 21 days demonstrated more staining for safranin-O, a marker of cartilage, compared to donor-matched conventionally isolated MSCs (FIG. 13C). Finally, when plated in adipogenic medium, elutriated/FACS sorted CD44− cells quickly acquired an adipogenic phenotype demonstrated by significant uptake of Oil Red O staining in lipid (FIG. 13D). Quantification of adipogenesis following isopropanol extraction of Oil Red O confirmed that CD44⁻ MSPCs formed more lipid compared to conventionally-isolated MSCs (FIG. 6D, far right) (P<0.034).

### Example 7

### CD44+MSPCs in Whole BM

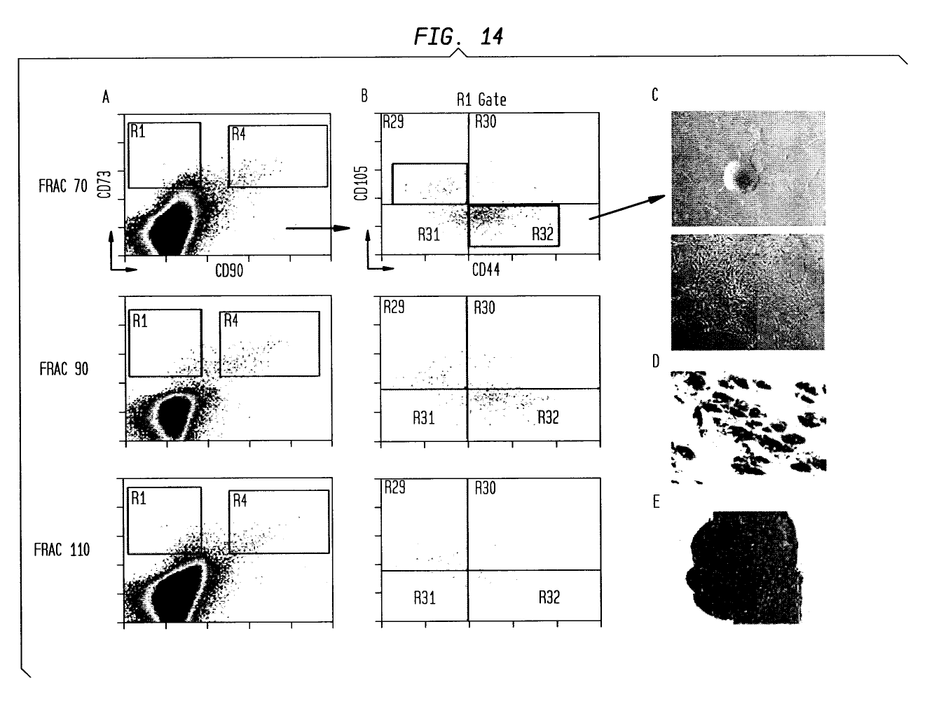

To investigate the presence of CD44⁺ cells in whole BM, back gating revealed a population of CD44+ cells that were positive for CD73 but that were negative for CD90. When the CD45⁻CD90⁻CD73⁺ gate (FIG. 14A, gate R1) was analyzed for CD44 and CD105 expression, a mixed population of cells was seen, predominantly either CD105⁺CD44⁻ (FIG. 14B, gate R29) or CD105⁻CD44⁺ (FIG. 14B, gate R32). Both of these populations were sorted, but only the CD105⁻CD44⁺ (gate 32) population was expandable. However, this population of cells, isolated from all elutriated fractions, displayed a dramatically different expansion capacity in culture compared to CD45⁻CD73⁺CD90⁺CD44⁻ cells. This CD44⁺ population grew very slowly as a spheroid in culture over a 21 day period (FIG. 14C, left), as opposed to the monolayer typically seen for cells with MSC activity (FIG. 14D, right). However, the spheroids could eventually be expanded, and after expansion displayed some evidence of a mesenchymal-like phenotype under defined conditions in vitro (FIG. 14D-E).

### Example 8

### Bone Regeneration Model

To test the ability of the isolated mesenchymal stem/progenitor cells (MSPCs) having a cell surface antigenic profile of CD34(−)/CD133(−)/CD45(−)/CD73(+)/CD90(+)/CD105(+)/CD44(−) to heal a bone defect, isolated mesenchymal stem/progenitor cells (MSPCs) are loaded into a ceramic carrier, and implanted into segmental bony defects in the femurs of adult arrhythmic rats, according to the methods described in Bruder, S. P. et al., “Bone regeneration by implantation of purified, culture-expanded human mesenchymal stem cells,” Journal of Orthopaedic Research, 16(2): 155-162 (1998), the entire disclosure of which is incorporated herein by reference. For comparison, cell-free ceramics are implanted in the contralateral limb. The animals are euthanized at 4, 8, or 12 weeks, and healing bone defects are compared by high-resolution radiography, immunohistochemistry, quantitative histomorphometry, and biomechanical testing.

### Example 9

### Spinal Cord Injury Model

The ability of the isolated mesenchymal stem/progenitor cells (MSPCs) of a cell surface antigenic profile CD34(−)/CD133(−)/CD45(−)/CD73(+)/CD90(+)/CD105(+)/CD44(−) to heal a spinal cord injury is tested using a spinal cord injury (SCI) mouse model, according to the methods described in Abematsu, M. et al., “Neurons derived from transplanted neural stem cells restore disrupted neuronal circuitry in a mouse model of spinal cord injury,” J. Clin. Invest., 120(9): 3255-3266 (2010), the entire disclosure of which is incorporated herein by reference. 15-week old male ICR mice are anesthetized, and laminectomies and partial laminectomies at the ninth and tenth thoracic spinal vertebrae are performed. The dorsal surface of the dura mater is exposed and SCI is applied using an SCI device. The muscle and skin are closed in layers. Seven days after injury, mice are anesthetized and receive the isolated mesenchymal stem/progenitor cells (MSPCs) and a carrier at the injury site of the injured spinal cord. For comparison, a cell-free carrier is implanted in a control group. Motor function of hind limbs are monitored up to 14 weeks after injury. The animals are euthanized at 14 weeks, and healing spinal cord injury is compared by immunohistochemistry, immunoelectron microscopy, etc.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

1. A composition comprising an isolated population of mesenchymal stem/progenitor cells (MSPCs), wherein the mesenchymal stem/progenitor cells (MSPCs) are of a cell surface antigenic profile CD34(−)/CD133(−)/CD45(−)/CD73(+)/CD90(+)/CD105(+)/CD44(−).

2. The composition according to claim 1, wherein at least 90% of the cells in the population are of the cell surface antigenic profile CD34(−)/CD133(−)/CD45(−)/CD73(+)/CD90(+)/CD105(+)/CD44(−).

3. The composition according to claim 1, wherein the composition further comprises a pharmaceutically acceptable carrier.

4. The composition according to claim 1, wherein size of the mesenchymal stem/progenitor cells (MSPCs) is from 5 μm to 10 μm.

5. The composition according to claim 1, wherein the mesenchymal stem/progenitor cells (MSPCs) are capable of being differentiated into ectodermal cells, mesodermal cells, or endodermal cells.

6. The composition according to claim 1, wherein the mesenchymal stem/progenitor cells (MSPCs) are capable of forming a three-dimensional spheroid.

7. The composition according to claim 5, wherein the ectodermal cells are capable of differentiation to neurons of a peripheral or central nervous system.

8. The composition according to claim 7, wherein the neurons express a neuronal marker β-Tubulin 3 (Tuj-1).

9. The composition according to claim 5, wherein the mesodermal cells are capable of differentiation to adipocytes, chondrocytes and osteoblasts.

10. The composition according to claim 9, wherein the mesodermal cells differentiated from the mesenchymal stem/progenitor cells (MSPCs) are capable of differentiation into ectodermal cells.

11. The composition according to claim 1, wherein the isolated mesenchymal stem/progenitor cells (MSPCs) can be expanded in a chemically defined medium.

12. The composition according to claim 1, wherein exposure of the mesenchymal stem/progenitor cells (MSPCs) to granulocyte-colony stimulating factor (G-CSF) results in appearance of a CD105(+)/CD44(+) cell population and a CD105(+)/CD44(−) cell population.

13. The composition according claim 1, wherein the mesenchymal stem/progenitor cell population of a cell surface antigenic profile CD34(−)/CD133(−)/CD45(−)/CD73(+)/CD90(+)/CD105(+)/CD44(−) is purified from cellular components of a bone marrow aspirate acquired from a subject.

14. The composition according to claim 1, wherein the mesenchymal stem/progenitor cell population of a cell surface antigenic profile CD34(−)/CD133(−)/CD45(−)/CD73(+)/CD90(+)/CD105(+)/CD44(−) is purified from peripheral blood.

15. The composition according to claim 14, wherein the mesenchymal stem/progenitor cells are capable of being mobilized by a stem cell mobilizing agent from the bone marrow into peripheral blood.

16. The composition according to claim 14, wherein the stem cell mobilizing agent is at least one of G-CSF, GM-CSF, and plerixafor (AMD3100).

17. The composition according to claim 1, wherein the mesenchymal stem/progenitor cell population of a cell surface antigenic profile CD34(−)/CD133(−)/CD45(−)/CD73(+)/CD90(+)/CD105(+)/CD44(−) is purified from umbilical cord blood.

18. A method for isolating and purifying a population of mesenchymal stem/progenitor cells (MSPCs) of a cell surface antigenic profile CD34(−)/CD133(−)/CD45(−)/CD73(+)/CD90(+)/CD105(+)/CD44(−), wherein the method is based on cluster of differentiation (CD) molecules on a surface of a pure initial population of cells, the method comprising:
(a) acquiring a source of CD34(−)/CD133(−)/CD45(−)/CD73(+)/CD90(+)/CD105(+)/CD44(−) cells from a mammal;
(b) depleting CD34-positive and CD133-positive cells from the cell source of (a) to obtain a first purified cell population of a cell surface antigenic profile CD34(−)/CD133(−);
(c) fractionating the first purified cell population of (b) using antibodies against cell surface antigens CD45, CD73, and CD90 to obtain a second purified cell population of the cell surface antigenic profile CD34(−)/CD133(−)/CD45(−)/CD73(+)/CD90(+); and
(d) further fractionating the second purified cell population of (c) using antibodies against cell surface antigens CD105 and CD44 to obtain a third purified cell population of the cell surface antigenic profile CD34(−)/CD133(−)/CD45(−)/CD73(+)/CD90(+)/CD105(+)/CD44(−);
wherein the method does not employ adherent culture of an unfractionated mononuclear cell population.

19. The method according to claim 18, wherein depletion of the CD34-positive and the CD133-positive cells in (a) is carried out by using a magnetic bead selection system.

20. The method according to claim 18, wherein steps (c) and (d) are performed with fluorescence-activated cell sorting (FACS).

21. The method according to claim 18, wherein the method further comprises (e) cryopreserving the third purified cell population by admixing the third purified cell population with a cryoprotectant and storing the population at low temperature.

22. The method according to claim 18, wherein the source of the CD34(−)/CD133(−)/CD45(−)/CD73(+)/CD90(+)/CD105(+)/CD44(−) cells is a bone marrow aspirate, a peripheral blood sample, or an umbilical cord.

23. The method according to claim 22, wherein the source of the CD34(−)/CD133(−)/CD45(−)/CD73(+)/CD90(+)/CD105(+)/CD44(−) cells is a bone marrow aspirate.

24. A method for obtaining an enriched population of mesenchymal stem/progenitor cells (MSPCs) using a size-based elutriation technique, wherein the size-based elutriation technique comprises flowing the sample obtained from a subject through a series of increasing flow rates in an elutriation device, and wherein each flow rate in the series of increasing flow rates collects a different population of cells in each flow rate fraction, the method comprising:
(1) acquiring a sample comprising CD34(−)/CD133(−)/CD45(−)/CD73(+)/CD90(+)/CD105(+)/CD44(−) cells from a mammal
(2) flowing the sample at a first flow rate,
wherein the first flow rate allows a first flow rate fraction comprising cells that are smaller than the mesenchymal stem/progenitor cells (MSPCs) in the sample to flow through the elutriation device and to be collected in a first cell collection bag of the elutriation device;
(3) increasing the first flow rate to a second flow rate,
wherein the second flow rate allows a second flow rate fraction comprising the mesenchymal stem/progenitor cells (MSPCs) in the sample to flow through the elutriation device and to be collected in a second cell collection bag of the elutriation device, and
wherein the second flow rate fraction collected in the second cell collection bag comprises the enriched population of the mesenchymal stem/progenitor cells (MSPCs);
(4) recirculating the sample comprising non-retained cells through the elutriation apparatus; and
(5) optionally increasing the second flow rate to a third flow rate, wherein the third flow rate allows a third flow rate fraction comprising cells that are larger than the mesenchymal stem/progenitor cells (MSPCs) in the sample to flow through the elutriation device and to be collected in a third cell collection bag of the elutriation device.

25. The method according to claim 24, wherein the first flow rate ranges from about 20 ml/minute to about 40 ml/minute, and wherein the first flow rate fraction comprises a substantial number of platelets.

26. The method according to claim 24, wherein the first flow rate is 20 ml/minute.

27. The method according to claim 24, wherein the first flow rate is 30 ml/minute.

28. The method according to claim 24, wherein the first flow rate is 40 ml/minute.

29. The method according to claim 24, wherein the second flow rate to obtain the enriched population of the mesenchymal stem/progenitor cells (MSPCs) ranges from about 50 ml/minute to about 90 ml/minute.

30. The method according to claim 29, wherein the second flow rate to obtain the enriched population of the mesenchymal stem/progenitor cells (MSPCs) is 50 ml/minute.

31. The method according to claim 29, wherein the second flow rate to obtain the enriched population of the mesenchymal stem/progenitor cells (MSPCs) is 70 ml/minute.

32. The method according to claim 29, wherein the second flow rate to obtain the enriched population of the mesenchymal stem/progenitor cells (MSPCs) is 90 ml/minute.

33. The method according to claim 24, wherein the third flow rate to obtain the third flow rate fraction comprising cells that are larger than the mesenchymal stem/progenitor cells (MSPCs) is greater than 90 ml/minute.

34. The method according to claim 24, wherein the third flow rate is greater than 90 ml/minute but less than 105 ml/minute.

35. The method according to claim 34, wherein the third flow rate that is greater than 90 ml/minute but less than 105 ml/minute collects a population of cells comprising Hematopoietic Stem Cells (HSCs).

36. The method according to claim 24, wherein the method further comprises removing undesired cells or components.

37. The method according to claim 22, wherein the method further comprises cryopreserving the enriched population of the mesenchymal stem/progenitor cells (MSPCs) by admixing the third purified cell population with a cryoprotectant and storing the cell population at a low temperature.

38. The method according to claim 24, further comprising:
(6) depleting CD34-positive and CD133-positive cells from the collected mesenchymal stem/progenitor cells (MSPCs) of (5) to obtain a first purified cell population of a cell surface antigenic profile CD34(−)/CD133(−);
(7) fractionating the first purified cell population of (6) using antibodies against cell surface antigens CD45, CD73, and CD90 to obtain a second purified cell population of the cell surface antigenic profile CD34(−)/CD133(−)/CD45(−)/CD73(+)/CD90(+); and
(8) further fractionating the second purified cell population of (7) using antibodies against cell surface antigens CD105 and CD44 to obtain a third purified cell population of the cell surface antigenic profile CD34(−)/CD133(−)/CD45(−)/CD73(+)/CD90(+)/CD105(+)/CD44(−);
wherein the method does not employ adherent culture of an unfractionated mononuclear cell population.

39. The method according to claim 18, wherein the isolated and purified population of mesenchymal stem/progenitor cells (MSPCs) of a cell surface antigenic profile CD34(−)/CD133(−)/CD45(−)/CD73(+)/CD90(+)/CD105(+)/CD44(−) are purified from cellular components of a bone marrow aspirate acquired from a subject.

40. The method according to claim 18, wherein the isolated population of mesenchymal stem/progenitor cells (MSPCs) of a cell surface antigenic profile CD34(−)/CD133(−)/CD45(−)/CD73(+)/CD90(+)/CD105(+)/CD44(−) is purified from peripheral blood.

41. The method according to claim 18, wherein the population of mesenchymal stem/progenitor cells (MSPCs) of a cell surface antigenic profile CD34(−)/CD133(−)/CD45(−)/CD73(+)/CD90(+)/CD105(+)/CD44(−) is purified from umbilical cord blood.

42. A method for differentiating a mesenchymal stem/progenitor cells (MSPCs) into a neuron-like cell population, the method comprising:
(a) culturing a population of mesenchymal stem/progenitor cells (MSPCs) of a cell surface antigenic profile CD34(−)/CD133(−)/CD45(−)/CD73(+)/CD90(+)/CD105(+)/CD44(−) in a chemically defined growth medium devoid of an animal serum;
(b) generating a spheroid by plating the mesenchymal stem/progenitor cells (MPCs) on a low-attachment plate;
(c) plating and culturing the mesenchymal stem/progenitor cells (MSPCs) from the spheroid in (b) in a defined medium for neuronal differentiation; and
(d) obtaining the neuron-like cell population differentiated from the mesenchymal stem/progenitor cells (MSPCs).

43. The method according to claim 42, wherein the neuron-like cell population expresses a neuronal marker β-Tubulin 3 (Tuj-1).

44. The method according to claim 42, wherein culturing in (c) is continued at least for 21 days.

45. A method for treating a degenerative condition or a diseased tissue condition in a subject, the method comprising:
(a) administering to the subject a therapeutically effective amount of the composition according to claim 1.

46. The method according to claim 45, wherein the degenerative condition or diseased tissue condition is a neurodegenerative disease, a neurological injury, a musculoskeletal defect, or a combination thereof.

47. The method according to claim 24, wherein the isolated and purified population of mesenchymal stem/progenitor cells (MSPCs) of a cell surface antigenic profile CD34(−)/CD133(−)/CD45(−)/CD73(+)/CD90(+)/CD105(+)/CD44(−) are purified from cellular components of a bone marrow aspirate acquired from a subject.

48. The method according to claim 22, wherein the isolated population of mesenchymal stem/progenitor cells (MSPCs) of a cell surface antigenic profile CD34(−)/CD133(−)/CD45(−)/CD73(+)/CD90(+)/CD105(+)/CD44(−) is purified from peripheral blood.

49. The method according to claim 22, wherein the population of mesenchymal stem/progenitor cells (MSPCs) of a cell surface antigenic profile CD34(−)/CD133(−)/CD45(−)/CD73(+)/CD90(+)/CD105(+)/CD44(−) is purified from umbilical cord blood.
